(19) United States
(12) Patent Application Publication
(10) Pub. No.: US 2024/0124561 A1
(43) Pub. Date: Apr. 18, 2024

(54) NUCLEIC ACIDS ENCODING HIV NEUTRALIZING ANTIBODIES AND USES THEREOF

(71) Applicant: MINICIRCLE INC.

(72) Inventors: Walter PATTERSON, Los Angeles, CA (US); Mac DAVIS, Los Angeles, CA (US)

(21) Appl. No.: 17/769,578

(22) PCT Filed: Oct. 16, 2020
(86) PCT No.: PCT/US2020/056081

§ 371 Date: Apr. 15, 2022

(51) Int. Cl. C07K 16/10; A61K 31/713; C12N 15/85

(52) U.S. Cl. CPC C07K 16/1063; A61K 31/713; C12N 15/85; C07K 2317/33; C07K 2317/76; C12N 2800/108

(57) ABSTRACT

Disclosed and described herein are nucleic acids encoding HIV broadly neutralizing antibodies. These antibodies are useful for treating or maintaining low viral loads in HIV infected individuals. The nucleic acids described herein are suitable for administration to individuals and provide long-lasting expression and secretion of the broadly neutralizing antibodies described into an individual's blood-stream.

### CROSS-REFERENCE

This application claims the benefit of U.S. Provisional Patent Application 62/916,732, filed on Oct. 17, 2019, and U.S. Provisional Patent Application 62/928,170 filed on Oct. 30, 2019, both of which are incorporated by reference herein in their entirety.

### BACKGROUND

As of 2018 nearly 40 million people globally were living with HIV, with over 1.7 million new infections that year. There is currently no reliable cure for HIV, and those living with the disease must continually take anti-retroviral therapies to maintain low viral loads. Anti-retroviral therapies are expensive, have side effects, and require daily attention to medications. There continues to be a need for therapeutic interventions with fewer side-effects and patient compliance issues.

### SUMMARY

Described herein are nucleic acids compositions encoding HIV specific broadly neutralizing antibodies, wherein the nucleic acid compositions possess the quality of long-lasting expression of the broadly neutralizing HIV antibodies in an individual.

One advantage of the compositions and methods described herein is the ability to treat multiple strains of HIV. Described herein are nucleic acids that encode broadly neutralizing antibodies to HIV. The nucleic acids described herein are suitable for administration to individuals and provide long-lasting expression and secretion of the broadly neutralizing antibodies described into an individual's blood-stream. As HIV is highly polymorphic, the compositions described herein may comprise a plurality of nucleic acids encoding different broadly neutralizing antibodies (or a single nucleotide encoding a plurality of different broadly neutralizing antibodies). Such combinations allow for an off-the-shelf treatment for individuals irrespective of the HIV strain that the individual is infected with.

Another advantage of the compositions and methods described herein is the long lasting and low-toxicity nature of the treatment. This advantage is due to the antibodies described herein being encoded by small nucleic acids known as minicircles and/or mini-intronic plasmids. Both of these types of plasmids are small and lack extraneous sequences of bacterial origin. As a result of their small size, the plasmids are less prone to transcriptional silencing by the cells that take up and express the plasmids leading to long-term expression. Additionally, while antibodies tend to have fewer side effects than small molecule treatments, these plasmids have low immunotoxicity owing to the reduction of immunostimulatory bacterial sequences.

In one aspect described herein is a nucleic acid encoding an HIV specific antibody, wherein the nucleic acid is a minicircle plasmid or a mini-intronic plasmid. In certain embodiments, the HIV specific antibody is a Fab, F(ab)₂, a single-domain antibody, or a single chain variable fragment (scFv). In certain embodiments, the HIV specific antibody is an IgG antibody. In certain embodiments, the HIV specific antibody is a broadly neutralizing HIV specific antibody. In certain embodiments, the HIV specific antibody binds to HIV gp120 and/or gp41. In certain embodiments, the broadly neutralizing antibody comprises 10E8.4, N6-LS, or PGDM1400. In certain embodiments, the broadly neutralizing antibody comprises 2F5, 4E10, 2G12, or B12. In certain embodiments, the broadly neutralizing antibody comprises N6-LS. In certain embodiments, the broadly neutralizing antibody comprises VRC07-523, CAP256-VRC26.25, 10-1074V, or 10E. In certain embodiments, the broadly neutralizing antibody comprises VRC07, VRC01, VRC13, CAP256-VRC26.08, PGT145, PG9, PGT121, or PGT128.

In certain embodiments, the minicircle plasmid or the mini-intronic plasmid comprises a S/MAR region. In certain embodiments, the nucleic acid is a component of a complex with a cationic lipid, a cationic polymer, or a nanodiamond. In certain embodiments, the nucleic acid is a component of a complex with a nanodiamond. In certain embodiments, the cationic lipid or cationic polymer comprises polyethylenimine, polylysine (PLL), PEG-grafted-polylysine (PEG-g-PLL), DOTMA, DOGS, DC-Chol, DEAE-dextran, DOSPA, DOPE, DOTAP, or combinations thereof. In certain embodiments, the cationic lipid or cationic polymer comprises polyethylenimine, polylysine (PLL), or PEG-grafted-polylysine (PEG-g-PLL). In certain embodiments, the nucleic acid or the complex is a component of a composition that further comprises a pharmaceutically acceptable carrier, diluent, or excipient. In certain embodiments, the composition is formulated for intravenous administration. In certain embodiments, the composition is formulated for intramuscular administration. In certain embodiments, the composition is formulated for subcutaneous administration. Also disclosed herein is a method of treating HIV infection in an individual comprising administering the nucleic acid, the complex, or the composition to the individual. Also disclosed herein is a method of preventing HIV infection in an individual comprising administering the nucleic acid, the complex, or the composition to the individual. Also disclosed herein is a method of maintaining an undetectable HIV viral load in the blood of an individual comprising administering the nucleic acid, the complex, or the composition to the individual. In certain embodiments the nucleic acid, the complex, or the composition is for use in treating or preventing an HIV infection in an individual. In certain embodiments the nucleic acid, the complex, or the composition is for use in maintaining undetectable HIV load in the blood of an individual. Also described herein is a method of making the minicircle nucleic acid, comprising inducing a site-specific recombinase or endonuclease in a prokaryotic host comprising a parental plasmid, wherein the parental plasmid comprises a nucleic acid encoding an HIV specific antibody and possesses a prokaryotic origin of replication, wherein the site-specific recombinase or endonuclease excises the prokaryotic origin of replication, resulting in a minicircle plasmid comprising the nucleic acid encoding the HIV specific antibody. In certain embodiments, the prokaryotic host is E. coli. Also described herein is a method of making the mini-intronic plasmid nucleic acid, comprising culturing a prokaryotic host comprising a mini-intronic plasmid, wherein the mini-intronic plasmid comprises a nucleic acid encoding an HIV specific antibody, wherein the nucleic acid encoding the HIV specific antibody is encoded by at least two exons, wherein an intron separating the at least two exons comprises a prokaryotic origin of replication and a selectable marker. In certain embodiments, the prokaryotic host is E. coli. In certain embodiments, the method further comprises purifying the minicircle plasmid or the mini-intronic plasmid from the prokaryotic host.

In another aspect described herein is a composition comprising a first nucleic acid and a second nucleic acid, wherein the first nucleic acid encodes a first HIV specific antibody that binds an HIV polypeptide, wherein the second nucleic acid encodes a second HIV specific antibody that binds an HIV polypeptide, wherein the amino acid sequence of the first HIV antibody is different from the amino acid sequence of the second HIV antibody. In certain embodiments, the composition comprises a third nucleic acid that encodes a third HIV specific antibody that specifically binds an HIV polypeptide, wherein the amino acid sequence of the third HIV antibody is different from the amino acid sequence of the first and second HIV antibody. In certain embodiments, any one or more of the first HIV specific antibody, second HIV specific antibody, or the third HIV specific antibody is a Fab, F(ab)₂, a single-domain antibody, or a single chain variable fragment (scFv). In certain embodiments, any one or more of the first HIV specific antibody, second HIV specific antibody, or the third HIV specific antibody is an IgG antibody. In certain embodiments, any one or more of the first HIV specific antibody, second HIV specific antibody, or the third HIV specific antibody is a broadly neutralizing HIV specific antibody. In certain embodiments, all three of the first HIV specific antibody, second HIV specific antibody, and the third HIV specific antibody are broadly neutralizing HIV specific antibodies. In certain embodiments, the HIV polypeptide is HIV gp120 and/or gp41. In certain embodiments, the first or second HIV specific antibody comprises any two of 10E8.4, N6-LS, or PGDM1400. In certain embodiments, the first or second HIV specific antibody comprises any two of 2F5, 4E10, 2G12 or B12. In certain embodiments, the first or second HIV specific antibody comprises any two of VRC07-523, CAP256-VRC26.25, 10-1074V, or 10E. In certain embodiments, the first or second HIV specific antibody comprises any two of VRC07, VRC01, VRC13, CAP256-VRC26.08, PGT145, PG9, PGT121, or PGT128. In certain embodiments, the third HIV specific antibody comprises 10E8.4, N6-LS, or PGDM1400, 2F5, 4E10, 2G12 B12, VRC07-523, CAP256-VRC26.25, 10-1074V, 10E VRC07, VRC01, VRC13, CAP256-VRC26.08, PGT145, PG9, PGT121, or PGT128, wherein the third HIV specific antibody is different from the first HIV specific antibody and the second HIV specific antibody. In certain embodiments, the first or second nucleic acid is a minicircle plasmid. In certain embodiments, the first and second nucleic acid is a minicircle plasmid. In certain embodiments, the first or second nucleic acid is a mini-intronic plasmid. In certain embodiments, the first and second nucleic acid is a mini-intronic plasmid. In certain embodiments, any one or more of the first, second or third nucleic acids are minicircle plasmids. In certain embodiments, any one or more of the first, second, or third nucleic acids are mini-intronic plasmids. In certain embodiments, the minicircle or mini-intronic plasmids comprise a S/MAR region. In certain embodiments, the first, second, and third nucleic are a component of a complex with a cationic lipid, a cationic polymer, or a nanodiamond. In certain embodiments, the nucleic acid is complexed to a nanodiamond. In certain embodiments, the cationic lipid or cationic polymer comprises polyethylenimine, polylysine (PLL), PEG-grafted-polylysine (PEG-g-PLL), DOTMA, DOGS, DC-Chol, DEAE-dextran, DOSPA, DOPE, DOTAP, or combinations thereof. In certain embodiments, the cationic lipid or cationic polymer comprises polyethylenimine, polylysine (PLL), or PEG-grafted-polylysine (PEG-g-PLL). In certain embodiments, the first, second, or third nucleic acids or the complex is a component of a composition further comprising a pharmaceutically acceptable carrier, diluent, or excipient. In certain embodiments, the composition is formulated for intravenous administration. In certain embodiments, the composition is formulated for intramuscular administration. In certain embodiments, the composition is formulated for subcutaneous administration. Also described herein is a method of treating HIV infection in an individual comprising administering the first, second, or third nucleic acids, the complex, or the composition. Also described herein is a method of preventing HIV infection in an individual comprising administering the first, second, or third nucleic acids, the complex, or the composition. Also described herein is a method of maintaining undetectable HIV viral load in the blood of an individual comprising administering the first, second, or third nucleic acids, the complex, or the composition. In certain embodiments, the first, second, or third nucleic acids are for use in treating or preventing an HIV infection in an individual. In certain embodiments, the first, second, or third nucleic acids are for use in maintaining undetectable HIV load in the blood of an individual. Also described herein is a method of making any of the first, second, or third nucleic acids comprising inducing a site-specific recombinase or endonuclease in a prokaryotic host comprising a parental plasmid, wherein the parental plasmid comprises a nucleic acid encoding an HIV specific antibody and possesses a prokaryotic origin of replication, wherein the site-specific recombinase or endonuclease excises the prokaryotic origin of replication, resulting in a minicircle plasmid comprising the nucleic acid encoding the HIV specific antibody. In certain embodiments, the prokaryotic host is E. coli. Also described herein is a method of making any of the first, second, or third nucleic acids, comprising culturing a prokaryotic host comprising a mini-intronic plasmid, wherein the mini-intronic plasmid comprises a nucleic acid encoding an HIV specific antibody, wherein the nucleic acid encoding the HIV specific antibody is encoded by at least two exons, wherein an intron separating the at least two exons comprises a prokaryotic origin of replication and a selectable marker. In certain embodiments, the prokaryotic host is E. coli. In certain embodiments, the method further comprises purifying the minicircle plasmid or the mini-intronic plasmid from the prokaryotic host.

### BRIEF DESCRIPTION OF THE DRAWINGS

FIG. 1A illustrates a vector map of a minicircle plasmid described herein comprising a nucleic acid sequence encoding the N6 antibody.

FIG. 1B illustrates a vector map of a mini-intronic plasmid described herein comprising a nucleic acid sequence encoding the N6 antibody.

FIG. 2A illustrates a dot blot of N6 transfected cell supernatants (1; neg. control; 2-4 supernatant from N6 transfected cells).

FIG. 2B illustrates Coomassie staining (left) and Western blot (right) of culture supernatant from N6 transfected cells (1; neg. control; 2-4 supernatant from N6 transfected cells).

FIG. 3A illustrates a dot blot of binding of N6 culture supernatant to immobilized HIV gp120.

FIG. 3B illustrates Coomassie staining (left) and Western blot (right) of HIV gp120 SDS-PAGE gels (run with 10, 100, or 1,000 picograms of gp120 as indicated) probed with N6 culture supernatant.

### DETAILED DESCRIPTION

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. Compositions for treating or preventing a given disease can consist essentially of the recited active ingredient, exclude additional active ingredients, but include other non-material components such as excipients, carriers, or diluents. “Consisting of” shall mean excluding more than trace elements of other ingredients and substantial method steps. Embodiments defined by each of these transition terms are within the scope of this disclosure.

As used herein the term “about” refers to an amount that is near the stated amount by 10%.

As used herein the terms “individual,” “patient,” or “subject” are used interchangeably and refer to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “treat” or “treating” refers to interventions to a physiological or disease state of an individual designed or intended to ameliorate at least one sign or symptom associated with said physiological or disease state. The skilled artisan will recognize that given a heterogeneous population of individuals afflicted with a disease, not all individuals will respond equally, or at all, to a given treatment. More specifically, as described herein treating refers to any administration of the compositions described herein to reduce HIV viral load in the plasma of an individual that has a detectable viral load, or to maintain a low viral load.

As used herein the term “prevent” or “preventing” refers to interventions designed to prevent HIV infection of an individual prophylactically. Compositions for prevention and methods of prevention are intended to be administered to an individual that is serum negative for the anti-HIV antibodies that mark infection. Additionally, individuals treated prophylactically can be HIV-positive and the administration is intended to prevent new infection with HIV virus from an exogenous source.

As used herein “broadly neutralizing” refers to an antibody that is able to bind and prevent cellular infection by at least three strains of HIV.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Among the provided antibodies are monoclonal antibodies and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements. One type of nucleic that can encode the antibodies described herein is a minicircle. As used herein a “minicircle” refers to a small circular double-stranded DNA molecule, generally less than about 5 kB, devoid of a bacterial origin of replication or antibiotic resistance cassette. The minicircles described herein comprise a eukaryotic promoter mated to a nucleotide sequence encoding a protein/antibody, and optionally, additional eukaryotic expression sequences such as a polyadenylation site or an enhancer sequence. Another type of nucleic acid that can encode the antibodies described herein is a mini-intronic plasmid. As used herein “mini-intronic plasmid” refers to a plasmid where the nucleotide sequence encoding an antibody is split into two or more exons that are divided by one or more introns. A bacterial origin of replication (e.g., PUC) and selectable marker (e.g., RNAOUT) is encoded within the one or more introns. Mini-intronic plasmids are described in Lu et al., “A Mini-intronic Plasmid (MW): A Novel Robust Transgene Expression Vector In Vivo and In Vitro.” Mol Ther. 2013 May; 21(5): 954-963.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

The term “pharmaceutically acceptable excipient,” as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention.

### Minicircles and Mini-Intronic Plasmids Encoding HIV Broadly Neutralizing Antibodies

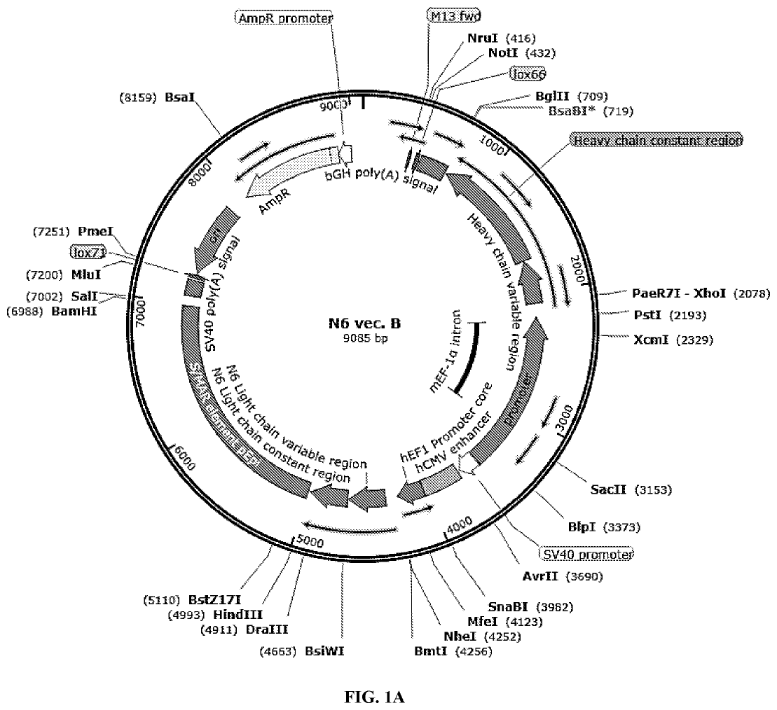

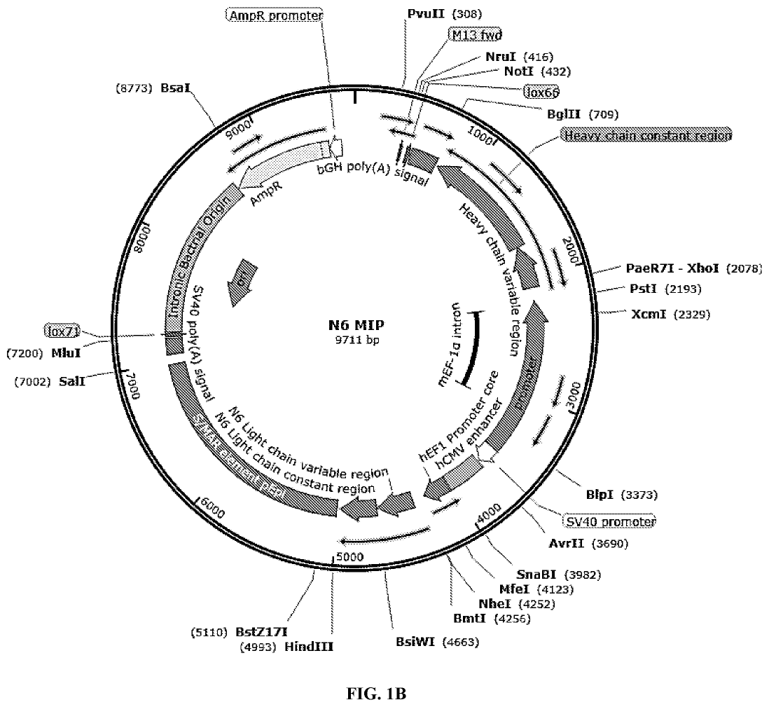

The nucleic acids encoding the HIV specific antibodies or HIV specific broadly neutralizing antibodies described herein are suitably minicircle or mini-intronic plasmids. Referring to FIG. 1A minicircle plasmids generally comprise or lack features distinct from standard bacterial plasmids. Primarily, recombination sites that flank bacterial sequences allow for the bacterial sequences to be removed using a recombinase. Exemplified in FIG. 1A are LOX sites able to be recombined by Cre recombinase, thus removing the intervening origin or replication and antibiotic resistance cassette. To facilitate removal of bacterial sequences from the parental plasmid (e.g., the full plasmid depicted), the parental plasmid is grown in a bacterial host that also expresses the recombinase. The resulting minicircle plasmid lacks the origin of replication, and selection marker. Minicircles may contain other features that are normally included in plasmids including promoter or enhancer elements, multiple cloning sites, or polyadenylation sites. In certain embodiments, the promoter that controls expression of one or more HIV specific antibodies may be an inducible promoter. In certain embodiments, the inducible promoter is a tetracycline inducible promoter. In certain embodiments, the minicircle further comprises a scaffold/matrix attachment region (S/MAR region), otherwise called SAR (scaffold-attachment region), or MAR (matrix-associated region). S/MAR regions are sequences in the DNA of eukaryotic chromosomes where the nuclear matrix attaches. Such regions further allow for sustained presence of the minicircle plasmid in a Eukaryotic cell. An exemplary minicircle plasmid comprising a coding region for the N6 HIV specific broadly neutralizing antibody is shown in SEQ ID NO: 1.

Referring to FIG. 1B mini-intronic plasmids differ from standard plasmids in several ways. Mini-intronic plasmids comprise an intron that comprises an intronic sequence which further comprises a bacterial intron. In certain embodiments, the intron is situated between two exons that together comprise a full coding region of an HIV specific broadly neutralizing antibody. In certain embodiments, the intron is situated between two exons that together comprise a full coding region of a selectable marker such as kanamycin, ampicillin, or RNAOUT. Mini-intronic plasmids may contain other features that are normally included in plasmids including promoter or enhancer elements, multiple cloning sites, or polyadenylation sites. In certain embodiments, the promoter that controls expression of one or more HIV specific antibodies may be an inducible promoter. In certain embodiments, the inducible promoter is a tetracycline inducible promoter. In certain embodiments, the mini-intronic plasmid further comprises a scaffold/matrix attachment region (S/MAR region), otherwise called SAR (scaffold-attachment region), or MAR (matrix-associated region). S/MAR regions are sequences in the DNA of eukaryotic chromosomes where the nuclear matrix attaches. Such regions further allow for sustained presence of the mini-intronic plasmid in a Eukaryotic cell. An exemplary mini-intronic plasmid comprising a coding region for the N6 HIV specific broadly neutralizing antibody is shown in SEQ ID NO: 2. An exemplary mini-intronic plasmid comprising a coding region for the N6 HIV specific broadly neutralizing antibody under the control of a tetracycline promoter is shown in SEQ ID NO: 3. An exemplary minicircle plasmid comprising a coding region for the PGDM14 HIV specific broadly neutralizing antibody is shown in SEQ ID NO: 5. An exemplary mini-intronic plasmid comprising a coding region for the PGDM14 HIV specific broadly neutralizing antibody is shown in SEQ ID NO: 5. An exemplary mini-intronic plasmid comprising a coding region for the PGDM14 HIV specific broadly neutralizing antibody under the control of a tetracycline promoter is shown in SEQ ID NO: 6. An exemplary minicircle plasmid comprising a coding region for the 10-1074 HIV specific broadly neutralizing antibody is shown in SEQ ID NO: 7.

In a certain aspect described herein is a nucleic acid encoding a broadly neutralizing HIV specific antibody, wherein the nucleic acid is a minicircle or a mini-intronic plasmid.

In another aspect described herein is a composition comprising a first nucleic acid and a second nucleic acid, wherein the first nucleic acid encodes a first HIV specific antibody that binds an HIV polypeptide, wherein the second nucleic acid encodes a second HIV specific antibody that binds an HIV polypeptide, wherein the amino acid sequence of the first HIV antibody is different from the amino acid sequence of the second HIV antibody. In certain embodiments, the first or second nucleic acids are minicircle plasmids. In certain embodiments, the first or second nucleic acids are mini-intronic plasmids.

In another aspect described herein is a composition comprising a first nucleic acid, a second nucleic acid, and a third nucleic acid, wherein the first nucleic acid encodes a first HIV specific antibody that binds an HIV polypeptide, wherein the second nucleic acid encodes a second HIV specific antibody that binds an HIV polypeptide, wherein the third nucleic acid encodes a third HIV specific antibody that binds an HIV polypeptide, wherein the amino acid sequence of the first, second, and third HIV antibody are different. In certain embodiments, any one or more of the first, second, or third nucleic acids are minicircle plasmids. In certain embodiments, any one or more of the first, second, or third nucleic acids are mini-intronic plasmids

In another aspect described herein is a composition comprising a minicircle plasmid, a second minicircle plasmid, and a third minicircle plasmid, wherein the first minicircle plasmid encodes a first HIV specific antibody that binds an HIV polypeptide, wherein the second minicircle plasmid encodes a second HIV specific antibody that binds an HIV polypeptide, wherein the third minicircle plasmid encodes a third HIV specific antibody that binds an HIV polypeptide, wherein the amino acid sequence of the first, second, and third HIV antibody are different.

In another aspect described herein is a composition comprising a first mini-intronic plasmid, a second mini-intronic plasmid, and a third mini-intronic plasmid, wherein the first mini-intronic plasmid encodes a first HIV specific antibody that binds an HIV polypeptide, wherein the second mini-intronic plasmid encodes a second HIV specific antibody that binds an HIV polypeptide, wherein the third mini-intronic plasmid encodes a third HIV specific antibody that binds an HIV polypeptide, wherein the amino acid sequence of the first, second, and third HIV antibody are different.

Also described herein are extend release nucleic acid compositions, comprising nucleic acids encoding a plurality of HIV specific broadly neutralizing antibodies. In certain embodiments, the plurality of HIV specific broadly neutralizing antibodies are at least 3 HIV specific broadly neutralizing antibodies.

The mini-intronic and minicircle plasmids are useful for gene therapy and for administration to individuals. Upon administration the plasmids may be maintained in a cell of the individual and lead to long term expression of mRNAs encoded by the plasmid, leading to long term translation and/or secretion of proteins or polypeptides transcribed from the mRNAs (e.g., extended release). Such expression can persist for at least about 1 week, 2 weeks, 3 weeks, or 4 weeks post administration. Such expression can persist for at least about 1 month, 2 months, 3 months, or 4 months, 6 months, 9 months or 12 months post administration. In certain embodiments, the extended release can be for at least 1 month. In certain embodiments, the extended release can be for at least 2 months. In certain embodiments, the extended release can be for at least 3 months. In certain embodiments, the extended release can be for at least 6 months.

### HIV Broadly Neutralizing Antibodies

As described herein the antibodies encoded by the nucleic acids include broadly neutralizing antibodies. HIV broadly neutralizing antibodies can be found for example at bnaber.org, which serves as an online repository for data on HIV broadly neutralizing antibodies. In certain, embodiments, the HIV broadly neutralizing antibody neutralizes greater than about 10%, 25%, 35%, 45%, or 50% of HIV strains based upon the TZM-PsV reporter assay as described in Sarzotti-Kelsoe et al., “Optimization and Validation of the TZM-bl Assay for Standardized Assessments of Neutralizing Antibodies Against HIV-1.” J Immunol Methods. 2014 July; 0: 131-146.

In certain embodiments, the HIV specific antibody encoded by the nucleic acid comprises one or more of 10-1074, 10E8, 10E8.4, 10E8v4-5R+100cF, 10E8VLS, 12A12, 12A21, 2F5, 2G12, 35022, 3BC176, 3BNC117, 3BNC55, 3BNC60, 3BNC62, 447-52D, 4E10, 5H/I1-BMV-D5, 8ANC195, b12, CAP256-VRC26.01, CAP256-VRC26.02, CAP256-VRC26.03, CAP256-VRC26.04, CAP256-VRC26.05, CAP256-VRC26.06, CAP256-VRC26.07, CAP256-VRC26.08, CAP256-VRC26.09, CAP256-VRC26.10, CAP256-VRC26.11, CAP256-VRC26.12, CAP256-VRC26.25, CH01, CH02, CH03, CH04, CH103, HGN194, HJ16, HK20, M66.6, NIH45-46, PCDN-33A, PCDN-33B, PCDN-38A, PG16, PG9, PGDM1400, PGDM1401, PGDM1402, PGDM1403, PGDM1404, PGDM1405, PGDM1406, PGDM1407, PGDM1408, PGDM1409, PGDM1410, PGDM1411, PGDM1412, PGT121, PGT122, PGT123, PGT125, BilA-SG, PGT126, PGT127, PGT128, PGT130, PGT131, PGT135, PGT136, PGT137, PGT141, PGT142, PGT143, PGT144, PGT145, PGT151, PGT152, VRC-CH30, VRC-CH31, VRC-CH32, VRC-CH33, VRC-CH34, VRC-PG04, VRC-PG04b, VRC-PG20, VRC01, VRC02, VRC03, VRC07, VRC07-523L, VRC23, or Z13, ibalizumab. In certain embodiments, the HIV specific antibody comprises 10E8.4, N6-LS, or PGDM1400. In certain embodiments, the HIV specific antibody comprises 2F5, 4E10, 2G12 or B12. In certain embodiments, the HIV specific antibody comprises N6-LS.

Certain amino acid sequences of HIV specific broadly neutralizing antibodies that can be encoded by the nucleic acids described herein are provided at the end of this disclosure.

Individuals that can be treated using the nucleic acids encoding the HIV specific antibodies described herein include HIV positive individuals that have never been treated, individuals that have been treated by one or more anti-retroviral drugs, individuals that have failed treatment with one or more anti-retroviral drugs, and individuals successfully treated with anti-retroviral drugs with an undetectable viral load using polymerase chain reaction detection of HIV mRNA.

The nucleic acids encoding broadly neutralizing antibodies described herein can also be used prophylactically to prevent HIV infection in certain individuals at high-risk of HIV exposure including sex workers, partners of HIV positive individuals, or individuals that have been exposed to possibly contaminated blood products.

The individual can be previously treated with an antiviral regimen. In certain embodiments, the individual can be selected for treatment based upon having an undetectable viral load or a viral load below 1000, 500, 200, 100, or 50 copies of HIV mRNA per milliliter. In certain embodiments, the individual has an undetectable viral load by a standard laboratory test such as polymerase chain reaction (PCR). In certain embodiments, the individual has a viral load (HIV mRNA) below 1000 copies/mL. In certain embodiments, the individual has a viral load below 500 copies/mL. In certain embodiments, the individual has a viral load below 200 copies/mL. In certain embodiments, the individual has a viral load below 100 copies/mL. In certain embodiments, the individual has a viral load below 50 copies/mL. In certain embodiments, the individual has a viral load below 1000, 500, 200, 100 or 50 copies/mL for at least 3 months, 6 months or a year before treatment with nucleic acids encoding broadly neutralizing HIV specific antibodies.

In certain embodiments, the individual to be treated using the nucleic acids encoding broadly neutralizing HIV antibodies described herein has previously been treated with an HIV anti-retroviral. In certain embodiments, the HIV anti-retroviral drug comprises or consists of Abacavir (Ziagen), Atazanavir (Reyataz), Darunavir (Prezista), Dolutegravir (Tivicay), Efavirenz (Sustiva), Elvitegravir, Emtricitabine (Emtriva), Etravirine (Intelence), Fosamprenavir (Telzir, Lexiva), Lamivudine (Epivir), Lopinavir/ritonavir (Kaletra), Maraviroc (Celsentri), Nevirapine (Viramune), Raltegravir (Isentress), Rilpivirine (Edurant), Ritonavir (Norvir), Tenofovir (Viread), Zidovudine (AZT, Retrovir) and combinations thereof. In certain embodiments, the HIV antiretroviral drug is a combination treatment comprising or consisting of, for example, Efavirenz/Emtricitabine/Tenofovir di soproxil fumarate (Atripla), Atazanavir/Cobicistat (Evotaz), Emtricitabine/Tenofovir (Descovy), Darunavir/Cobicistat (Rezolsta), Elvitegravir/Cobicistat/Emtricitabine/Tenofovir (Stribild), Abacavir/Dolutegravir/Lamivudine (Triumeq), Emtricitabine/rilpivirine/Tenofovir (Odefsey), Rilpivirine/Emtricitabine/Tenofovir (Eviplera), Abacavir/Lamivudine (Kivexa), and Elvitegravir/Cobicistat/Emtricitabine/Tenofovir (Genvoya).

The nucleic acids encoding the HIV specific antibodies described herein can be further complexed with a compound that promotes entry of the nucleic acids into cells after administration to an individual. In particular, cationic compounds promote fusion with negatively charged cell membranes and entry of nucleic acids into a cell. Cationic compounds that can be used with the current invention include N-[1-(2,3-dioleyloxy)propyl]-N,N,N-trimethylammonium chloride(DOTMA), [1,2-bis(oleoyloxy)-3-(trimethylammonio)propane] (DOTAP), 3β[N—(N′, N′-dimethylaminoethane)-carbamoyl] cholesterol (DC-Chol), dioctadecylamidoglycylspermine (DOGS), and polyethylenimine.

Dioleoylphosphatidylethanolamine (DOPE), a neutral lipid, can used in conjunction with cationic lipids because of its membrane destabilizing effects at low pH, which aide in endolysosomal escape. In certain embodiments, the nucleic acids described herein are complexed with a cationic lipid or a cationic polymer. In certain embodiments, the cationic lipid or cationic polymer comprises polylysine (PLL), PEG-grafted-polylysine (PEG-g-PLL), polyethylenimine (PEG-g-PLL), DOTMA, DC-Chol, DOGS, DEAE-dextran, DOSPA, DOPE, DOTAP, or combinations thereof. In certain embodiments, the cationic lipid or cationic polymer comprises polyethylenimine. In certain embodiments, the cationic lipid or cationic polymer comprises polylysine (PLL). In certain embodiments, the cationic lipid or cationic polymer comprises PEG-grafted-polylysine (PEG-g-PLL). In certain embodiments, the nucleic acids described herein can be delivered using diamond nanoparticles (e.g., nanodiamonds). Complexes can then be delivered by intravenous, intramuscular, or subcutaneous injection. Alternatively, the nucleic acids described herein can be administered by non-cationic lipid-based methods common in gene therapy including electroporation, surface electroporation, or gene gun.

Once minicircles and mini-intronic plasmids are delivered to cells they are episomally maintained and resist degradation and transcriptional silencing. Dosages can thus be applied once monthly, once every two-months, once every three months, or once every four months. The first one, two, three, four, five or more initial doses can be administered more frequently in order to increase plasma levels of broadly neutralizing HIV specific antibodies, followed by maintenance doses administered monthly, every other month, once every two months, once every three months, or once every four months. Alternatively, patients can be monitored at weekly or monthly intervals using an anti-idiotype antibody that recognizes an HIV specific broadly neutralizing antibody. If the amount of an HIV specific broadly neutralizing antibody falls below about 50 ug/mL, 40 ug/mL, 30 ug/mL, 20 ug/mL, 10 ug/mL, or 5 ug/mL in the plasma of an individual, then the individual can receive an additional dose of a minicircle or mini-intronic plasmid composition encoding the HIV specific broadly neutralizing antibodies described herein.

Nucleic acids encoding HIV specific broadly neutralizing antibodies can be administered in naked form or complexed with a compound that aides entry into human cells. In certain embodiments, the compound comprises polylysine (PLL), PEG-grafted-polylysine (PEG-g-PLL), polyethylenimine (PEG-g-PLL), DOTMA, DC-Chol, DOGS, DEAE-dextran, DOSPA, DOPE, DOTAP, or combinations thereof. In some embodiments, an individual receives a dosage of nucleic acid encoding an HIV specific broadly neutralizing antibody equivalent to about 1 microgram to about 500 micrograms. In some embodiments, an individual receives a dosage of nucleic acid encoding an HIV specific broadly neutralizing antibody equivalent to about 1 microgram to about 2 micrograms, about 1 microgram to about 5 micrograms, about 1 microgram to about 10 micrograms, about 1 microgram to about 25 micrograms, about 1 microgram to about 50 micrograms, about 1 microgram to about 100 micrograms, about 1 microgram to about 250 micrograms, about 1 microgram to about 500 micrograms, about 1 microgram to about 1 microgram, about 2 micrograms to about 5 micrograms, about 2 micrograms to about 10 micrograms, about 2 micrograms to about 25 micrograms, about 2 micrograms to about 50 micrograms, about 2 micrograms to about 100 micrograms, about 2 micrograms to about 250 micrograms, about 2 micrograms to about 500 micrograms, about 2 micrograms to about 1 microgram, about 5 micrograms to about 10 micrograms, about 5 micrograms to about 25 micrograms, about 5 micrograms to about 50 micrograms, about 5 micrograms to about 100 micrograms, about 5 micrograms to about 250 micrograms, about 5 micrograms to about 500 micrograms, about 5 micrograms to about 1 microgram, about 10 micrograms to about 25 micrograms, about 10 micrograms to about 50 micrograms, about 10 micrograms to about 100 micrograms, about 10 micrograms to about 250 micrograms, about 10 micrograms to about 500 micrograms, about 10 micrograms to about 1 microgram, about 25 micrograms to about 50 micrograms, about 25 micrograms to about 100 micrograms, about 25 micrograms to about 250 micrograms, about 25 micrograms to about 500 micrograms, about 25 micrograms to about 1 microgram, about 50 micrograms to about 100 micrograms, about 50 micrograms to about 250 micrograms, about 50 micrograms to about 500 micrograms, about 50 micrograms to about 1 microgram, about 100 micrograms to about 250 micrograms, about 100 micrograms to about 500 micrograms, about 100 micrograms to about 1 microgram, about 250 micrograms to about 500 micrograms, about 250 micrograms to about 1 microgram, or about 500 micrograms to about 1 microgram. In some embodiments, an individual receives a dosage of nucleic acid encoding an HIV specific broadly neutralizing antibody equivalent to about 1 microgram, about 2 micrograms, about 5 micrograms, about 10 micrograms, about 25 micrograms, about 50 micrograms, about 100 micrograms, about 250 micrograms, about 500 micrograms, or about 1 microgram. In some embodiments, an individual receives a dosage of nucleic acid encoding an HIV specific broadly neutralizing antibody equivalent to at least about 1 microgram, about 2 micrograms, about 5 micrograms, about 10 micrograms, about 25 micrograms, about 50 micrograms, about 100 micrograms, about 250 micrograms, or about 500 micrograms. In some embodiments, an individual receives a dosage of nucleic acid encoding an HIV specific broadly neutralizing antibody equivalent to at most about 2 micrograms, about 5 micrograms, about 10 micrograms, about 25 micrograms, about 50 micrograms, about 100 micrograms, about 250 micrograms, about 500 micrograms, or about 1 microgram. The nucleic acids delivered in the composition or as a naked vector may be a minicircle, a mini-intronic plasmid, or any combination thereof. Dosages of nucleic acids may vary based upon the resulting efficiency with which the nucleic acids are taken up and expressed by cells. Such efficiency relies upon the exact method of administration is used, and which compounds are used to promote uptake and expression in human cells.

Also described herein are methods of expressing one or more HIV broadly neutralizing antibodies in a cell of an individual comprising administering one or more minicircle or mini-intronic plasmids encoding the HIV broadly neutralizing antibodies to an individual. Also described herein are methods of expressing two or more HIV broadly neutralizing antibodies in a cell of an individual comprising administering one or more minicircle or mini-intronic plasmids encoding the HIV broadly neutralizing antibodies to an individual. Also described herein are methods of expressing three or more HIV broadly neutralizing antibodies in a cell of an individual comprising administering one or more minicircle or mini-intronic plasmids encoding the HIV broadly neutralizing antibodies to an individual.

Also described herein are methods of expressing one or more HIV broadly neutralizing antibodies in a cell of an individual for at least three months post administration comprising administering one or more minicircle or mini-intronic plasmids encoding the HIV broadly neutralizing antibodies to an individual. Also described herein are methods of expressing two or more HIV broadly neutralizing antibodies in a cell of an individual for at least three months post administration comprising administering one or more minicircle or mini-intronic plasmids encoding the HIV broadly neutralizing antibodies to an individual. Also described herein are methods of expressing three or more HIV broadly neutralizing antibodies in a cell of an individual for at least three months post administration comprising administering one or more minicircle or mini-intronic plasmids encoding the HIV broadly neutralizing antibodies to an individual.

Also described herein are methods of expressing one or more HIV broadly neutralizing antibodies in a cell of an individual for at least six months post administration comprising administering one or more minicircle or mini-intronic plasmids encoding the HIV broadly neutralizing antibodies to an individual. Also described herein are methods of expressing two or more HIV broadly neutralizing antibodies in a cell of an individual for at least six months post administration comprising administering one or more minicircle or mini-intronic plasmids encoding the HIV broadly neutralizing antibodies to an individual. Also described herein are methods of expressing three or more HIV broadly neutralizing antibodies in a cell of an individual for at least six months post administration comprising administering one or more minicircle or mini-intronic plasmids encoding the HIV broadly neutralizing antibodies to an individual.

### Methods of Making

Described herein in one aspect is a method of making a minicircle encoding an HIV specific antibody, the method comprising inducing a site-specific recombinase or endonuclease in a prokaryotic host comprising a parental plasmid, wherein the parental plasmid comprises a nucleic acid encoding an HIV specific antibody and possesses a prokaryotic origin of replication, wherein the site-specific recombinase or endonuclease excises the prokaryotic origin of replication, resulting in a minicircle plasmid comprising the nucleic acid encoding the HIV specific antibody. In certain embodiments, the minicircle is further purified from the eukaryotic host.

Described herein in another aspect is a method of making a nucleic acid encoding an HIV specific antibody, the method comprising culturing a prokaryotic host cell comprising a mini-intronic plasmid, wherein the mini-intronic plasmid comprises a nucleic acid encoding an HIV specific antibody, wherein the nucleic acid encoding the HIV specific antibody is encoded by at least two exons, wherein an intron separating the at least two exons comprises a prokaryotic origin of replication and a selectable marker. In certain embodiments, the mini-intronic plasmid is further purified from the eukaryotic host using standard purification methods or methods consistent with producing nucleic acid for administration to an individual (e.g. low-endotoxin, <5 EU/kg).

Methods of purifying and isolating plasmids from a prokaryotic host are known in the art and can be applied to minicircle and mini-intronic plasmids described herein.

### Pharmaceutically Acceptable Excipients, Carriers, and Diluents

In certain embodiments, the nucleic acids of the current disclosure are included in a pharmaceutical composition comprising one or more pharmaceutically acceptable excipients, carriers, and diluents. These excipients, carriers, or diluents are in addition to the nucleic acids which may be complexed with the cationic lipids or cationic polymers described herein. In certain embodiments, the nucleic acids of the current disclosure are administered suspended in a sterile solution. In certain embodiments, the solution comprises about 0.9% NaCl or about 5% glucose or dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the nucleic acids of the current disclosure are shipped/stored lyophilized and reconstituted before administration. In certain embodiments, lyophilized nucleic acid formulations comprise a bulking agent such as, mannitol, sorbitol, sucrose, trehalose, dextran 40, or combinations thereof. The lyophilized formulation can be contained in a vial comprised of glass or other suitable non-reactive material. The nucleic acids when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

For injection, one or more agents can be formulated in aqueous solutions, including but not limited to physiologically compatible buffers such as Hank's solution, Ringer's solution, or physiological saline buffer. Such compositions can also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P.

One or more agents can also be formulated as a depot preparation. Such long acting formulations can be administered by implantation or transcutaneous delivery (for example subcutaneously or intramuscularly), intramuscular injection or use of a transdermal patch. Thus, for example, one or more agents can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising one or more agents can exert local and regional effects when administered topically or injected at or near particular sites of infection. Direct topical application, e.g., of a viscous liquid, gel, jelly, cream, lotion, ointment, suppository, foam, or aerosol spray, can be used for local administration, to produce, for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983.

Also described herein are kits comprising one or more of the nucleic acids encoding the HIV antibodies described herein in a suitable container and one or more additional components selected from: instructions for use; a diluent, an excipient, a carrier, and a device for administration.

In certain embodiments, described herein is a method of preparing a HIV treatment comprising admixing one or more pharmaceutically acceptable excipients, carriers, or diluents and one or more nucleic acids encoding the HIV antibodies of the current disclosure. In certain embodiments, described herein is a method of preparing an HIV treatment for storage or shipping comprising lyophilizing one or more nucleic acids encoding antibodies of the current disclosure.

Described are certain specific numbered embodiments below:
1. A nucleic acid encoding an HIV specific antibody, wherein the nucleic acid is a minicircle plasmid or a mini-intronic plasmid.
2. The nucleic acid of embodiment [0080]1, wherein the HIV specific antibody is a Fab, F(ab)₂, a single-domain antibody, or a single chain variable fragment (scFv).
3. The nucleic acid of embodiment [0080]1 or [0080]2, wherein the HIV specific antibody is an IgG antibody.
4. The nucleic acid of any one of embodiments [0080]1 to [0080]3, wherein the HIV specific antibody is a broadly neutralizing HIV specific antibody.
5. The nucleic acid of any one of embodiments [0080]1 to [0080]4, wherein the HIV specific antibody binds to HIV gp120 and/or gp41.
6. The nucleic acid of any one of embodiments [0080]1 to [0080]5, wherein the broadly neutralizing antibody comprises 10E8.4, N6-LS, or PGDM1400.
7. The nucleic acid of any one of embodiments [0080]1 to [0080]5, wherein the broadly neutralizing antibody comprises 2F5, 4E10, 2G12, or B12.
8. The nucleic acid of any one of embodiments [0080]1 to [0080]5, wherein the broadly neutralizing antibody comprises N6-LS.
9. The nucleic acid of any one of embodiments [0080]1 to [0080]5, wherein the broadly neutralizing antibody comprises VRC07-523, CAP256-VRC26.25, 10-1074V, or 10E.
10. The nucleic acid of any one of embodiments [0080]1 to [0080]5, wherein the broadly neutralizing antibody comprises VRC07, VRC01, VRC13, CAP256-VRC26.08, PGT145, PG9, PGT121, or PGT128.
11. The nucleic acid of any one of embodiments [0080]1 to [0080]10, wherein the minicircle plasmid or the mini-intronic plasmid comprises a S/MAR region.
12. A complex comprising the nucleic acid of any one of embodiments [0080]1 to [0080]11 complexed to a cationic lipid, a cationic polymer, or a nanodiamond.
13. The complex of embodiment [0080]12, wherein the nucleic acid of any one of embodiments [0080]1 to [0080]11 is complexed to a nanodiamond.
14. The complex of embodiments [0080]13, wherein the cationic lipid or cationic polymer comprises polyethylenimine, polylysine (PLL), PEG-grafted-polylysine (PEG-g-PLL), DOTMA, DOGS, DC-Chol, DEAE-dextran, DOSPA, DOPE, DOTAP, or combinations thereof.
15. The complex of embodiments [0080]14, wherein the cationic lipid or cationic polymer comprises polyethylenimine, polylysine (PLL), or PEG-grafted-polylysine (PEG-g-PLL).
16. A composition comprising the nucleic acid of any one of embodiments [0080]1 to [0080]11 or the complex of any one of embodiments [0080]12 to [0080]15 and a pharmaceutically acceptable carrier, diluent, or excipient.
17. The composition of embodiment [0080]16, formulated for intravenous administration.
18. The composition of embodiment [0080]16, formulated for intramuscular administration.
19. The composition of embodiment [0080]16, formulated for subcutaneous administration.
20. A method of treating HIV infection in an individual comprising administering the nucleic acid of any one of embodiments [0080]1 to [0080]11, the complex of any one of embodiments [0080]12 to [0080]15, or the composition of any one of embodiments [0080]16 to [0080]19 to the individual.
21. A method of preventing HIV infection in an individual comprising administering the nucleic acid of any one of embodiments [0080]1 to [0080]11, the complex of any one of embodiments [0080]12 to [0080]15, or the composition of any one of embodiments [0080]16 to [0080]19 to the individual.
22. A method of maintaining undetectable HIV load in the blood of an individual comprising administering the nucleic acid of any one of embodiments [0080]1 to [0080]11, the complex of any one of embodiments [0080]12 to [0080]15, or the composition of any one of embodiments [0080]16 to [0080]19 to the individual.
23. The nucleic acid of any one of embodiments [0080]1 to [0080]11, the complex of any one of embodiments [0080]12 to [0080]15, or the composition of any one of embodiments [0080]16 to [0080]19 for use in treating or preventing an HIV infection in an individual.
24. The nucleic acid of any one of embodiments [0080]1 to [0080]11, the complex of any one of embodiments [0080]12 to [0080]15, or the composition of any one of embodiments [0080]16 to [0080]19 for use in maintaining undetectable HIV load in the blood of an individual.
25. A method of making the nucleic acid of any one of embodiments [0080]1 to [0080]11, comprising inducing a site-specific recombinase or endonuclease in a prokaryotic host comprising a parental plasmid, wherein the parental plasmid comprises a nucleic acid encoding an HIV specific antibody and possesses a prokaryotic origin of replication, wherein the site-specific recombinase or endonuclease excises the prokaryotic origin of replication, resulting in a minicircle plasmid comprising the nucleic acid encoding the HIV specific antibody.
26. A method of making the nucleic acid of any one of embodiments [0080]1 to [0080]11, comprising culturing a prokaryotic host comprising a mini-intronic plasmid, wherein the mini-intronic plasmid comprises a nucleic acid encoding an HIV specific antibody, wherein the nucleic acid encoding the HIV specific antibody is encoded by at least two exons, wherein an intron separating the at least two exons comprises a prokaryotic origin of replication and a selectable marker.
27. The method of embodiments [0080]25 or [0080]26, wherein the prokaryotic host is E. coli.
28. The method of any one of embodiments [0080]25 to [0080]27, further comprising purifying the minicircle plasmid or the mini-intronic plasmid from the prokaryotic host.
29. A composition comprising a first nucleic acid and a second nucleic acid, wherein the first nucleic acid encodes a first HIV specific antibody that binds an HIV polypeptide, wherein the second nucleic acid encodes a second HIV specific antibody that binds an HIV polypeptide, wherein the amino acid sequence of the first HIV antibody is different from the amino acid sequence of the second HIV antibody.
30. The composition according to embodiment [0080]29, wherein the composition comprises a third nucleic acid that encodes a third HIV specific antibody that specifically binds an HIV polypeptide, wherein the amino acid sequence of the third HIV antibody is different from the amino acid sequence of the first and second HIV antibody.
31. The composition of embodiments [0080]29 or [0080]30, wherein any one or more of the first HIV specific antibody, second HIV specific antibody, or the third HIV specific antibody is a Fab, F(ab)₂, a single-domain antibody, or a single chain variable fragment (scFv).
32. The composition of embodiments [0080]29 or [0080]30, wherein any one or more of the first HIV specific antibody, second HIV specific antibody, or the third HIV specific antibody is an IgG antibody.
33. The composition of any one of embodiments [0080]29 to [0080]32, wherein any one or more of the first HIV specific antibody, second HIV specific antibody, or the third HIV specific antibody is a broadly neutralizing HIV specific antibody.
34. The composition of any one of embodiments [0080]29 to [0080]33, wherein all three of the first HIV specific antibody, second HIV specific antibody, and the third HIV specific antibody are broadly neutralizing HIV specific antibodies.
35. The composition of any one of embodiments [0080]29 to [0080]34, wherein the HIV polypeptide is HIV gp120 and/or gp41.
36. The composition of any one of embodiments [0080]29 to [0080]34, wherein the first or second HIV specific antibody comprises any two of 10E8.4, N6-LS, or PGDM1400.
37. The composition of any one of embodiments [0080]29 to [0080]34, wherein the first or second HIV specific antibody comprises any two of 2F5, 4E10, 2G12 or B12.
38. The composition of any one of embodiments [0080]29 to [0080]34, wherein the first or second HIV specific antibody comprises any two of VRC07-523, CAP256-VRC26.25, 10-1074V, or 10E.
39. The composition of any one of embodiments [0080]29 to [0080]34, wherein the first or second HIV specific antibody comprises any two of VRC07, VRC01, VRC13, CAP256-VRC26.08, PGT145, PG9, PGT121, or PGT128.
40. The composition of any one of embodiments [0080]30 to [0080]39, wherein the third HIV specific antibody comprises 10E8.4, N6-LS, or PGDM1400, 2F5, 4E10, 2G12 B12, VRC07-523, CAP256-VRC26.25, 10-1074V, 10E VRC07, VRC01, VRC13, CAP256-VRC26.08, PGT145, PG9, PGT121, or PGT128, wherein the third HIV specific antibody is different from the first HIV specific antibody and the second HIV specific antibody.
41. The composition of any one of embodiments [0080]29 to [0080]40, wherein the first or second nucleic acid is a minicircle plasmid.
42. The composition of any one of embodiments [0080]29 to [0080]40, wherein the first and second nucleic acid is a minicircle plasmid.
43. The composition of any one of embodiments [0080]29 to [0080]40, wherein the first or second nucleic acid is a mini-intronic plasmid.
44. The composition of any one of embodiments [0080]29 to [0080]40, wherein the first and second nucleic acid is a mini-intronic plasmid.
45. The composition of any one of embodiments [0080]30 to [0080]40, wherein any one or more of the first, second or third nucleic acids are minicircle plasmids.
46. The composition of any one of embodiments [0080]30 to [0080]40, wherein any one or more of the first, second, or third nucleic acids are mini-intronic plasmids.
47. The composition of any one of embodiments [0080]41 to [0080]46, wherein the minicircle or mini-intronic plasmids comprise a S/MAR region.
48. A complex comprising the first, second, and third nucleic acids of any one of embodiments [0080]29 to [0080]47 complexed to a cationic lipid, a cationic polymer, or a nanodiamond.
49. The complex of embodiment [0080]48, wherein the nucleic acid of any one of embodiments [0080]29 to [0080]47 is complexed to a nanodiamond.
50. The complex of embodiment [0080]49, wherein the cationic lipid or cationic polymer comprises polyethylenimine, polylysine (PLL), PEG-grafted-polylysine (PEG-g-PLL), DOTMA, DOGS, DC-Chol, DEAE-dextran, DOSPA, DOPE, DOTAP, or combinations thereof.
51. The complex of embodiment [0080]50, wherein the cationic lipid or cationic polymer comprises polyethylenimine, polylysine (PLL), or PEG-grafted-polylysine (PEG-g-PLL).
52. A composition comprising the first, second, or third nucleic acids of any one of embodiments [0080]29 to [0080]47 or the complex of any one of embodiments [0080]48 to [0080]51 and a pharmaceutically acceptable carrier, diluent, or excipient.
53. The composition of embodiment [0080]52, formulated for intravenous administration.
54. The composition of embodiment [0080]52, formulated for intramuscular administration.
55. The composition of embodiment [0080]52, formulated for subcutaneous administration.
56. A method of treating HIV infection in an individual comprising administering the first, second, or third nucleic acids of any one of embodiments [0080]29 to [0080]47 or the complex of any one of embodiments [0080]48 to [0080]51, or the composition of any one of embodiments [0080]52 to [0080]55 to the individual.
57. A method of preventing HIV infection in an individual comprising administering the first, second, or third nucleic acids of any one of embodiments [0080]29 to [0080]47 or the complex of any one of embodiments [0080]48 to [0080]51, or the composition of any one of embodiments [0080]52 to [0080]55 to the individual.
58. A method of maintaining undetectable HIV load in the blood of an individual comprising administering the first, second, or third nucleic acids of any one of embodiments [0080]29 to [0080]47 or the complex of any one of embodiments [0080]48 to [0080]51, or the composition of any one of embodiments [0080]52 to [0080]55 to the individual.
59. The first, second, or third nucleic acids of any one of embodiments [0080]29 to [0080]47 or the complex of any one of embodiments [0080]48 to [0080]51, or the composition of any one of embodiments [0080]52 to [0080]55 for use in treating or preventing an HIV infection in an individual.
60. The first, second, or third nucleic acids of any one of embodiments [0080]29 to [0080]47 or the complex of any one of embodiments [0080]48 to [0080]51, or the composition of any one of embodiments [0080]52 to [0080]55 for use in maintaining undetectable HIV load in the blood of an individual.
61. A method of making any of the first, second, or third nucleic acids of any one of embodiments [0080]29 to [0080]47, comprising inducing a site-specific recombinase or endonuclease in a prokaryotic host comprising a parental plasmid, wherein the parental plasmid comprises a nucleic acid encoding an HIV specific antibody and possesses a prokaryotic origin of replication, wherein the site-specific recombinase or endonuclease excises the prokaryotic origin of replication, resulting in a minicircle plasmid comprising the nucleic acid encoding the HIV specific antibody.
62. A method of making any of the first, second, or third nucleic acids of any one of embodiments [0080]29 to [0080]47, comprising culturing a prokaryotic host comprising a mini-intronic plasmid, wherein the mini-intronic plasmid comprises a nucleic acid encoding an HIV specific antibody, wherein the nucleic acid encoding the HIV specific antibody is encoded by at least two exons, wherein an intron separating the at least two exons comprises a prokaryotic origin of replication and a selectable marker.
63. The method of embodiments [0080]61 or [0080]62, wherein the prokaryotic host is E. coli.
64. The method of any one of embodiments [0080]61 to [0080]63, further comprising purifying the minicircle plasmid or the mini-intronic plasmid from the prokaryotic host.

### EXAMPLES

The following illustrative examples are representative of embodiments of compositions and methods described herein and are not meant to be limiting in any way.

### Example 1—Production of a Broadly Neutralizing HIV Antibody from Human Cells Using Minicircles

Transient expression of a broadly neutralizing HIV antibody (N6) was tested in Expi293F cells.

### Materials

Cells: Epxi293F, ThermoFisher Scientific Cat #A14527; Medium: Expi293™ Expression Medium, ThermoFisher Scientific Cat #A1435101; ExpiFectamine™ 293 Transfection Kit: ThermoFisher Scientific Cat #A14525; Culture vessel: Corning polycarbonate Erlenmeyer flask with vent cap, various capacities; N6 plasmid: reconstituted in TE, concentration determined by Nanodrop ND-1000; HRP conjugated Anti-human IgG (H+L): Jackson Immuno Research Laboratories Cat #109-035-088; Purified human IgG: Jackson Immuno Research Laboratories Cat #009-000-002

### Protocol

Ten ml of healthy Epxi293F cells were grown in 125 ml flasks and transfected with purified plasmid using ExpiFectamine™ 293 transfection reagent by following a procedure recommended by the vendor. Three identical cultures were transfected with N6 plasmid. One flask of cells was not transfected as a negative control. The transfected cells were sampled 3, 4, 5, and 6 days post-transfection. On the 6thday post-transfection the media of transfected cells as well as control cultures were centrifuged for 10 minutes at 2,000×g at 4° C. Clarified media were transferred to a new tube. To the clarified supernatant 0.1% sodium azide was added as a preservative. The supernatants were stored at 4° C.

### Analysis

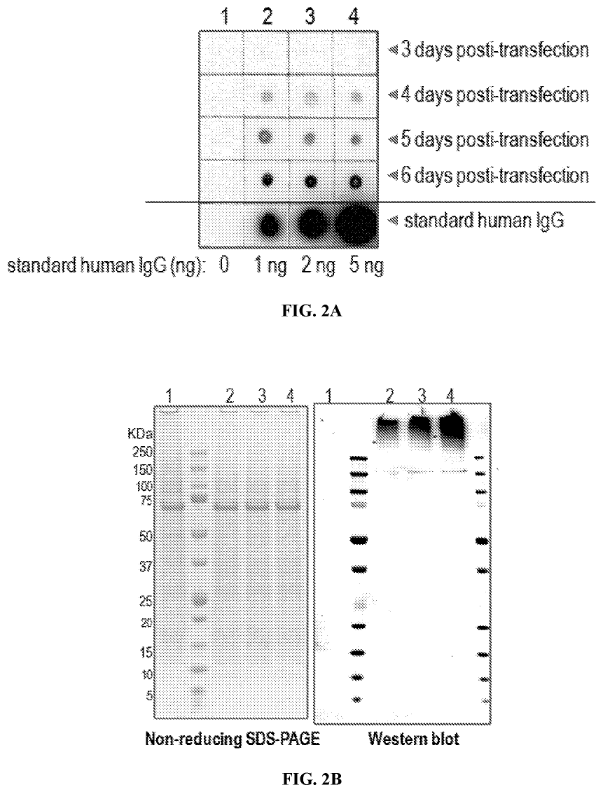

The level of recombinant antibody secreted by the cells in the culture medium was analyzed by Dot blot using HRP conjugated goat anti-human IgG (H+L) as a detection antibody. As shown in FIG. 2A, by dot blot analysis the amount of N6 antibody secreted by transfected cells increased with time indicating that N6 antibody is made and secreted from human cells. Confirmed by Coomassie stain and western blot as shown in FIG. 2B.

### Example 2—Functional Testing of Broadly Neutralizing HIV Antibodies Produced from Human Cells Using Minicircles

Antibodies produced as in Example 1 were tested for their ability to bind HIV gp120 antigen.

### Materials

gp120 antigen: Native Antigen Cat #HIVCM244-GP120-100, 100 μg of lyophilized antigen was reconstituted with 0.50 ml of 50% glycerol and stored at −20° C.; N6 antibody: N6 culture supernatant from example 1; HRP conjugated Anti-human IgG (H+L): Jackson Immuno Research Laboratories Cat #109-035-088 LDS sample buffer (4×): ThermoFisher Scientific Cat #NP0007

### Protocol

### Dot Blot

Varying amounts of native gp120 antigen was printed on nitrocellulose filters. Filters were blocked with 5% fetal bovine serum and 0.05% Tween 20 in phosphate buffer saline (PBS). Filters were incubated with N6 culture supernatant, which was either non-diluted or diluted with 0.05% Tween 20 in PBS. After overnight incubation at 4° C. filters were washed 4 times with 0.05% Tween20 in PBS. Filters were incubated with 10,000-fold diluted HRP conjugated goat anti-human IgG (H+L), and dot blots were developed using HRP chemiluminescent substrate.

### Western Blot

gp120 antigen was treated under both reducing and non-reducing conditions. Reducing treatment was as follows: gp120 antigen was mixed with LDS sample buffer (final 1×) supplemented with 50 mM DTT (final concentration) and then heated for 10 minutes at 70° C. Non-reducing treatment was as follows: gp120 antigen was mixed with LDS sample buffer (final 1×). The sample was not heated. Both reduced and non-reduced gp120 were resolved by SDS-PAGE, transferred to nitrocellulose, blocked, and incubated with N6 culture supernatant, which was diluted 4-fold with 0.05% Tween in PBS. After overnight incubation at 4° C. filters were washed with 0.05% Tween in PBS, incubated with 10,000-fold diluted HRP conjugated goat anti-human IgG (H+L), and developed using HRP chemiluminescent substrate.

### Analysis

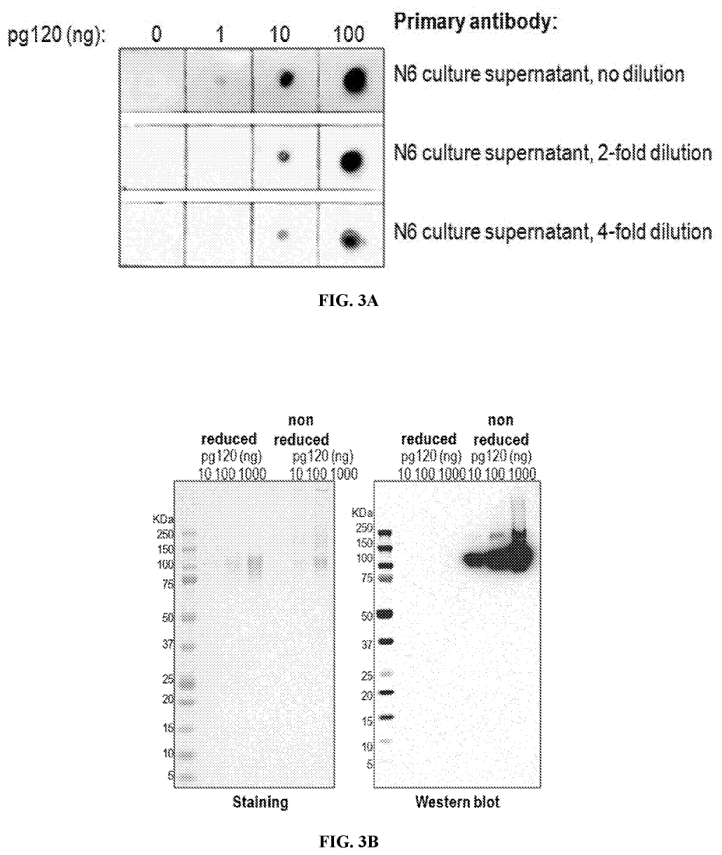

As shown in FIG. 3A, the N6 antibody is able to recognize native gp120. However, as shown in FIG. 3B, N6 antibody does not recognize fully denatured and reduced gp120, but does recognize partially denatured non-reduced gp120. These results indicate that human cells expressing a broadly neutralizing HIV antibody produce functional antibody that binds native HIV gp120.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequences described herein
SEQ ID NO 1
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTCGGTACC
TCGCGAATGCATCTAGAgcggccgcataacttcgtatagcatacattatacgaacggtaACCTGGGTACCCATA
GAGCCCACCGCATCCCCAGCATGCCTGCTATTGTCTTCCCAATCCTCCCCCTTGCTGTCCTGCCCCACCCCACC
CCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCATTTACCtggAGACAGGGAGAGGCTCTTCTGCGTGTAGTGG
TTGTGCAGAGCCTCATGCATCACGGAGCATGAGAAGACGTTCCCCTGCTGCCACCTGCTCTTGTCCACG
GTGAGCTTGCTGTAGAGGAAGAAGGAGCCatcagaGTCCAGCACGGGAGGCGTGGTCTTGTAGTTGTTCT
CCGGCTGCCCATTGCTCTCCCACTCCACagcGATGTCGCTGGGATAGAAGCCTTTGACCAGGCAGGTCA
GGCTGACCTGGTTCTTGGTCAGCTCATCCCGGGATGGGGGCAGGGTGTACACCTGTGGTTCTCGGGGCT
GCCCTTTGGCTTTGGAGATGGTTTTCTCGATGGGGGCTGGGAGGGCTTTGTTgctGACCTTGCACTTGTA
CTCCTTGCCATTCAGCCAGTCCTGGTGCAGGACGGTGAGGACactGACCACACGGTACGTGCTGTTGTAC
TGCTCCTCcctCGGCTTTGTCTTGGCATTATGCACCTCCACtccatcCACGTACCAGTTGAACTTGACCTCAG
GGTCTTCatgGCTCACatcCACCACCACGCATGTGACCTCAGGGGTCCGGGAGATCATGAGGGTGTCCTTG
GGTTTTGGGGGGAAGAGGAAGACTGACGGTCCCCCCAGGAGTTCAGGTGCTGGGCACGGTGGGCATG
TGTGAGTTTTGTCACAAGATTTGGGCTCAACTTTCTTGTCCACCTTGGTGTTGCTGGGCTTGTGATTCAC
GTTGCAGATGTAGGTCTGGGTGCCCAAGCTGCTGGAGGGCACGGTCACCACGCTGCTGAGGGAGTAGA
GTCCTGAGGACTGTAGGACAGCCGGGAAGGTGTGCACGCCGCTGGTCAGGGCtccTGAGTTCCACGACA
CagtCACCGGTTCGGGGAAGTAGTCCTTGACCAGGCAGCCCAGGGCCGCTGTGCCCCCAGAGGTGCTCT
TGGAGGAGGGTGCCAGGGGGAAGACactTGGGCCCTTGGTactCGCCGCagaGACGACGACagtCGTTCCCT
GTCCCCAGGCATCTAAGGCCCAAGAGGAGTCtccATAGGAcctatcTCTCGCACAGTAATAGACagcCGTGTC
atcAGGTTTAAGGCCTCTGATGTCCATGTACGCAATCTCTCTATATACatcTCGtgtCAATGTGACCCTGTCC
CGAAAACCACCACCAAAATTCACagcCCCATATTGTGGCTTGATCCACCCCACCCACTCAAGTCCTCGCC
CaggGGCCTGTCGGAACCAAAATAATATGTGGGCGGTAAAGGTGTATCCAGAGGTCTGGCAGGAGACT
CTTACTGAGGCtccCGGTTTCTTCATCGCAGTCCCTGATTGTACCAGGTGCGCTCGAGAATGTACACCGG
TTGCAGTTGCTACTAGAAAAAGGATGATACATGACCATCCCATGGTGGTCTAGtGCaGttaCGATATCTGA
TCACACGTGTCGAGGACaGTGACTGCAGATTGCTTTGAATTAGCGGTGGTTTTCACAACACCTAAAAAA
GGGTTTAAAAGATACCTTTGAACCGCTAAGAAGCCCGAGAATTAGCTCCGCTCAAAACTCAAGGGGAC
AAATTCCAAAAATGACTTCCAGCGCCAGGCTGGCCTGACTAGTCTCCACCCACCAAATGTGAACAAAC
TCCAACGCCATTACATCCCCTCCCCCCGCCGCGACTAGCCGTGCTCAAAAGCCCGAGGTGACTATTGCG
GCCGATAGGACCACGGGGTCACAGGAAGCAGCAGCCGGTGAGGGACCAGGCCCTCTTCCTTTGTGTGG
GTGACTCACCCGCCCGCTCCACCGGGCTGCCGCGTCCTCCATTTTGAGCTCCTTGCAACAGGGCCCGGG
AGCGGCCATCTTTCCACGCACGCAACTGGTGCCGGACGGGATGGCCTCACCCTAGTTAGGGAGGCAGG
GCAACGCGGCGCCGCCAAGCCAGATCGTGCCGGGTGCTGGGGCCACATGGCCTCGGCACGCTAACCCC
AGCCTGGTTGCTTCGGGAAAAACCCCAGGCCTCGCCCCATCCAGGTGGCGTCGGACATGTGCTCCGAA
GGCGGGCGGGCCCCAGCCGCCACTCCTGTCCCTCCATTCCTCCCCAACCATGACCTCTCCGGGCTCCGG
GCGAGCAAGCCCCGCACCCTCCCTTTGTTAGCCCCTATTGCTGAACGGCAATCGAAGGCAGCAGGGCA
ACAACAACAAAAAAAAAAAAGACCAGAGTGCGGCCGGAGTAGCACGCGGCGGCGGCGCGGACACCA
CGCTAGGCCTCAAGCCGGACACGAGGCGAGGCTACGGGGTTGCCGCTAGGCCTCGCACTCTGCCTCCC
GCGCCGCCCGCAACTCGAAGCGGGAATGCTCGCAGCTAATCCCCGCCGACGACAGCGGGGCCCGGCC
CGCTCGGAGCAGGACCTCCAGCTCGGCGGCCCGCGGAAGCCACACCCGCCCCTCACCTGCGTTCTGAC
GGCAAACCCGTTGCGAAAAAGAACGTCCAAGGCGACTGCCGCACTTATATACCGTTCTCCCCCACCCT
CGGGAAGAGGGCGGAGCCAGCACACGACACCGCTTTCCCAGTTTGCCCCGCGCCAGCTCCCCGGTCCT
CCCCCCCACGACCAGGGTCTATGAGCTTGTGTGCTCTGCTCTCCCCGCGCTCAGCCCTGAGCGCATGCT
CCTCGCGCTGTCGTGGGGCGGCTACAGGGATTTGGGTCTGTGAGAATTTAGGGACGGTCCCTGGGCTC
TCCCCTTGCGAAGGCGATCCCTCCTTTTGTATGAATTACTCTCGGCTCCACTAGTGGGCCCTGCAaGgaatt
ctGTTAGGGTGTGGAAAGTCCCCAGGCTCCCCAGCAGGCAGAAGTATGCAAAGCATGCATCTCAATTAG
TCAGCAACCAGGTGTGGAAAGTCCCCAGGCTCCCCAGCAGGCAGAAGTATGCAAAGCATGCATCTCAA
TTAGTCAGCAACCATAGTCCcctaggGAGACATGTTTACATAACTTACGGTAAATGGCCCGCCTGGCTGAC
CGCCCAACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGTACAT
GACCTTATGGGACTTTCCTACTTGGCAGTACATCTACGTATTAGTCATCGCTATTACCATGAATTGGTTT
GATCTGATTATAACTAGTGGAGAAGAGCATGCTTGAGGGCTGAGTGCCCCTCAGTGGGCAGAGAGCAC
ATGGCCCACAGTCCCTGAGAAGTTGGGGGGAGGGGTGGGCAATTGAACTGGTGCCTAGAGAAGGTGG
GGCTTGGGTAAACTGGGAAAGTGATGTGGTGTACTGGCTCCACCTTTTTCCCCAGGGTGGGGGAGAAC
CATATATAAGTGCAGTAGTCTCTGTGAACATTCGCTAGCACACGTGTGATCAGATATCatGGCtGCTCTtA
GACCACCATGGGATGGTCATGTATCATCCTTTTTCTAGTAGCAACTGCAACCGGTGTACATTCATACAT
CCACGTGACCCAGTCTCCGTCCTCCCTGTCTGTGTCTATTGGAGACAGAGTCACCATCaacTGCCAGACG
AGTCAGGGTGTTGGCAGTGACCTACATTGGTATCAACACAAACCGGGGAGAGCCCCTAAACTCTTGAT
CCACCATACCTCTTCTGTGGAAGACGGTGTCCCCTCAAGATTCAGCGGCTCTGGATTTCACACATCTTT
TAATCTGACCATCAGCGACCTACAGGCTGACGACATTGCCACATATTACTGTCAAGTTTTACAAttctttGG
CCGAGGGAGTagaCTCCATATTAAACGTACGGTGGCTGCACCATCTGTCTTCATCTTCccaCCATCTGATG
AGCAGTTGAAATCTGGAACTGCCTCTGTTGTGTGCCTGCTGAATAACTTCTACCCCAGAGAAGCCAAA
GTGCAGTGGAAGGTGGACaatGCCctcCAGAGCGGAAACAGCCAGGAAtctGTGACAGAGCAGGATTCCAA
GGATTCCACATACAGCCTGAGCAGCACACTGACACTGTCCAAGgctGACTACGAGAAGCACAAGGTGTA
CGCCTGCGAAGTGACACACCAGGGACTGTCCTCCCCTGTGACAAAGAGCTTCAACAGAGGAGAATGCT
GATAGGATaagctttGATCTAAATAAACTTATAAATTGTGAGAGAAATTAATGAATGTCTAAGTTAATGCA
GAAACGGAGAGACATACTATATTCATGAACTAAAAGACTTAATATTGTGAAGGTATACTTTCTTTCCAC
ATAAATTTGTAGTCAATATGTTCACCCCAAAAAAGCTGTTTGTTAACTTGCCAACCTCATTCTAAAATG
TATATAGAAGCCCAAAAGACAATAACAAAAATATTCTTGTAGAACAAAATGGGAAAGAATGTTCCACT
AAATATCAAGATTTAGAGCAAAGCATGAGATGTGTGGGGATAGACAGTGAGGCTGATAAAATAGAGT
AGAGCTCAGAAACAGACCCATTGATATATGTAAGTGACCTATGAAAAAAATATGGCATTTTACAATGG
GAAAATGATGATCTTTTTCTTTTTTAGAAAAACAGGGAAATATATTTATATGTAAAAAATAAAAGGGA
ACCCATATGTCATACCATACACACAAAAAAATTCCAGTGAATTATAAGTCTAAATGGAGAAGGCAAAA
CTTTAAATCTTTTAGAAAATAATATAGAAGCATGCCATCATGACTTCAGTGTAGAGAAAAATTTCTTAT
GACTCAAAGTCCTAACCACAAAGAAAAGATTGTTAATTAGATTGCATGAATATTAAGACTTATTTTTAA
AATTAAAAAACCATTAAGAAAAGTCAGGCCATAGAATGACAGAAAATATTTGCAACACCCCAGTAAA
GAGAATTGTAATATGCAGATTATAAAAAGAAGTCTTACAAATCAGTAAAAAATAAAACTAGACAAAA
ATTTGAACAGATGAAAGAGAAACTCTAAATAATCATTACACATGAGAAACTCAATCTCAGAAATCAGA
GAACTATCATTGCATATACACTAAATTAGAGAAATATTAAAAGGCTAAGTAACATCTGTGGCAATATT
GATGGTATATAACCTTGATATGATGTGATGAGAACAGTACTTTACCCCATGGGCTTCCTCCCCAAACCC
TTACCCCAGTATAAATCATGACAAATATACTTTAAAAACCATTACCCTATATCTAACCAGTACTCCTCA
AAACTGTCAAGGTCATCAAAAATAAGAAAAGTCTGAGGAACTGTCAAAACTAAGAGGAACCCAAGGA
GACATGAGAATTATATGTAATGTGGCATTCTGAATGAGATCCCAGAACAGAAAAAGAACAGTAGCTA
AAAAACTAATGAAATATAAATAAAGTTTGAACTTTAGTTTTTTTTAAAAAAGAGTAGCATTAACACGG
CAAAGCCATTTTCATATTTTTCTTGAACATTAAGTACAAGTCTATAATTAAAAATTTTTTAAATGTAGTC
TGGAACATTGCCAGAAACAGAAGTACAGCAGCTATCTGTGCTGTCGCCTAACTATCCATAGCTGATTG
GTCTAAAATGAGATACATCAACGCTCCTCCATGTTTTTTGTTTTCTTTTTAAATGAAAAACTTTATTTTT
TAAGAGGAGTTTCAGGTTCATAGCAAAATTGAGAGGAAGGTACATTCAAGCTGAGGAAGTTTTCCTCT
ATTCCTAGTTTACTGAGAGATTGCATCATGAATGGGTGTTAAATTTTGTCAAATGCTTTTTCTGTGTCTA
TCAATATGACCATGTGATTTTCTTCTTTAACCTGTTGATGGGACAAATTACGTTAATTGATTTTCAAACG
TTGAACCACCCTTACATATCTGGAATAAATTCTACTTGGTTGTGGTGTATATTTTTTGATACATTCTTGG
ATTCTTTTTGCTAATATTTTGTTGAAAATGTTTGTATCTTTGTTCATGAGAGATATTGGTCTGTTGTTTTC
TTTTCTTGTAATGTCATTTTCTAGTTCCGGTATTAAGGTAATGCTGGCCTAGTTGAATGATTTAGGAAGT
ATTCCCTCTGCTTCTGTCTTCTGAAAGAGATTGTAGAAAGTTGATACAATTTTTTTTTCTTTAAATATCTTG
ATAGAATTggatcctatcaatcGTCGACtGTACCttGGGCCtGGGACCGGAACTGAAACATAAAATGAATGCA
AcTGTTGTTGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCATGTCTG
GATCACGCGTAGtctagataccgttcgtatagcatacattatacgaagttatgtttaaacagCTGGCGTTTTT
CCATAGGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGACCGCTGCGCCTTA
TCCGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAAAGAGTT
GGTAGCTCTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCAGTGGAA
CGAAAACTCACGTTAAGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGTTACCAATGCTT
AATCAGTGAGGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCGAGACCCAC
GCTCACCGGCTCCAGATTTATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTT
TTACTTTCACCAGCGTTTCTGGGTGAGCAAAAACAGGAAGGCAAAATGCCGCAAAAAAGGGAATAAG
GGCGACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGCACAT
TTCCCCGAAAAGTGCCACCTGACGTCTAAGAAACCATTATTATCATGACATTAACCTATAAAAATAGG
CGTATCACGAGGCCCTTTCGTC
SEQ ID NO: 2
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTCGGTAC
CTCGCGAATGCATCTAGAgcggccgcataacttcgtatagcatacattatacgaacggtaACCTGGGTACCCA
TAGAGCCCACCGCATCCCCAGCATGCCTGCTATTGTCTTCCCAATCCTCCCCCTTGCTGTCCTGCCCCACCCC
ACCCCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCATTTACCtggAGACAGGGAGAGGCTCTTCTGCGTGTAGTGG
TTGTGCAGAGCCTCATGCATCACGGAGCATGAGAAGACGTTCCCCTGCTGCCACCTGCTCTTGTCCACG
GTGAGCTTGCTGTAGAGGAAGAAGGAGCCatcagaGTCCAGCACGGGAGGCGTGGTCTTGTAGTTGTTCT
CCGGCTGCCCATTGCTCTCCCACTCCACagcGATGTCGCTGGGATAGAAGCCTTTGACCAGGCAGGTCA
GGCTGACCTGGTTCTTGGTCAGCTCATCCCGGGATGGGGGCAGGGTGTACACCTGTGGTTCTCGGGGCT
GCCCTTTGGCTTTGGAGATGGTTTTCTCGATGGGGGCTGGGAGGGCTTTGTTgctGACCTTGCACTTGTA
CTCCTTGCCATTCAGCCAGTCCTGGTGCAGGACGGTGAGGACactGACCACACGGTACGTGCTGTTGTAC
TGCTCCTCcctCGGCTTTGTCTTGGCATTATGCACCTCCACtccatcCACGTACCAGTTGAACTTGACCTCAG
GGTCTTCatgGCTCACatcCACCACCACGCATGTGACCTCAGGGGTCCGGGAGATCATGAGGGTGTCCTTG
GGTTTTGGGGGGAAGAGGAAGACTGACGGTCCCCCCAGGAGTTCAGGTGCTGGGCACGGTGGGCATG
TGTGAGTTTTGTCACAAGATTTGGGCTCAACTTTCTTGTCCACCTTGGTGTTGCTGGGCTTGTGATTCAC
GTTGCAGATGTAGGTCTGGGTGCCCAAGCTGCTGGAGGGCACGGTCACCACGCTGCTGAGGGAGTAGA
GTCCTGAGGACTGTAGGACAGCCGGGAAGGTGTGCACGCCGCTGGTCAGGGCtccTGAGTTCCACGACA
CagtCACCGGTTCGGGGAAGTAGTCCTTGACCAGGCAGCCCAGGGCCGCTGTGCCCCCAGAGGTGCTCT
TGGAGGAGGGTGCCAGGGGGAAGACactTGGGCCCTTGGTactCGCCGCagaGACGACGACagtCGTTCCCT
GTCCCCAGGCATCTAAGGCCCAAGAGGAGTCtccATAGGAcctatcTCTCGCACAGTAATAGACagcCGTGTC
atcAGGTTTAAGGCCTCTGATGTCCATGTACGCAATCTCTCTATATACatcTCGtgtCAATGTGACCCTGTCC
CGAAAACCACCACCAAAATTCACagcCCCATATTGTGGCTTGATCCACCCCACCCACTCAAGTCCTCGCC
CaggGGCCTGTCGGAACCAAAATAATATGTGGGCGGTAAAGGTGTATCCAGAGGTCTGGCAGGAGACT
CTTACTGAGGCtccCGGTTTCTTCATCGCAGTCCCTGATTGTACCAGGTGCGCTCGAGAATGTACACCGG
TTGCAGTTGCTACTAGAAAAAGGATGATACATGACCATCCCATGGTGGTCTAGtGCaGttaCGATATCTGA
TCACACGTGTCGAGGACaGTGACTGCAGATTGCTTTGAATTAGCGGTGGTTTTCACAACACCTAAAAAA
GGGTTTAAAAGATACCTTTGAACCGCTAAGAAGCCCGAGAATTAGCTCCGCTCAAAACTCAAGGGGAC
AAATTCCAAAAATGACTTCCAGCGCCAGGCTGGCCTGACTAGTCTCCACCCACCAAATGTGAACAAAC
TCCAACGCCATTACATCCCCTCCCCCCGCCGCGACTAGCCGTGCTCAAAAGCCCGAGGTGACTATTGCG
GCCGATAGGACCACGGGGTCACAGGAAGCAGCAGCCGGTGAGGGACCAGGCCCTCTTCCTTTGTGTGG
GTGACTCACCCGCCCGCTCCACCGGGCTGCCGCGTCCTCCATTTTGAGCTCCTTGCAACAGGGCCCGGG
AGCGGCCATCTTTCCACGCACGCAACTGGTGCCGGACGGGATGGCCTCACCCTAGTTAGGGAGGCAGG
GCAACGCGGCGCCGCCAAGCCAGATCGTGCCGGGTGCTGGGGCCACATGGCCTCGGCACGCTAACCCC
AGCCTGGTTGCTTCGGGAAAAACCCCAGGCCTCGCCCCATCCAGGTGGCGTCGGACATGTGCTCCGAA
GGCGGGCGGGCCCCAGCCGCCACTCCTGTCCCTCCATTCCTCCCCAACCATGACCTCTCCGGGCTCCGG
GCGAGCAAGCCCCGCACCCTCCCTTTGTTAGCCCCTATTGCTGAACGGCAATCGAAGGCAGCAGGGCA
ACAACAACAAAAAAAAAAAAGACCAGAGTGCGGCCGGAGTAGCACGCGGCGGCGGCGCGGACACCA
CGCTAGGCCTCAAGCCGGACACGAGGCGAGGCTACGGGGTTGCCGCTAGGCCTCGCACTCTGCCTCCC
GCGCCGCCCGCAACTCGAAGCGGGAATGCTCGCAGCTAATCCCCGCCGACGACAGCGGGGCCCGGCC
CGCTCGGAGCAGGACCTCCAGCTCGGCGGCCCGCGGAAGCCACACCCGCCCCTCACCTGCGTTCTGAC
GGCAAACCCGTTGCGAAAAAGAACGTCCAAGGCGACTGCCGCACTTATATACCGTTCTCCCCCACCCT
CGGGAAGAGGGCGGAGCCAGCACACGACACCGCTTTCCCAGTTTGCCCCGCGCCAGCTCCCCGGTCCT
CCCCCCCACGACCAGGGTCTATGAGCTTGTGTGCTCTGCTCTCCCCGCGCTCAGCCCTGAGCGCATGCT
CCTCGCGCTGTCGTGGGGCGGCTACAGGGATTTGGGTCTGTGAGAATTTAGGGACGGTCCCTGGGCTC
TCCCCTTGCGAAGGCGATCCCTCCTTTTGTATGAATTACTCTCGGCTCCACTAGTGGGCCCTGCAaGgaatt
ctGTTAGGGTGTGGAAAGTCCCCAGGCTCCCCAGCAGGCAGAAGTATGCAAAGCATGCATCTCAATTAG
TCAGCAACCAGGTGTGGAAAGTCCCCAGGCTCCCCAGCAGGCAGAAGTATGCAAAGCATGCATCTCAA
TTAGTCAGCAACCATAGTCCcctaggGAGACATGTTTACATAACTTACGGTAAATGGCCCGCCTGGCTGAC
CGCCCAACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGTACAT
GACCTTATGGGACTTTCCTACTTGGCAGTACATCTACGTATTAGTCATCGCTATTACCATGAATTGGTTT
GATCTGATTATAACTAGTGGAGAAGAGCATGCTTGAGGGCTGAGTGCCCCTCAGTGGGCAGAGAGCAC
ATGGCCCACAGTCCCTGAGAAGTTGGGGGGAGGGGTGGGCAATTGAACTGGTGCCTAGAGAAGGTGG
GGCTTGGGTAAACTGGGAAAGTGATGTGGTGTACTGGCTCCACCTTTTTCCCCAGGGTGGGGGAGAAC
CATATATAAGTGCAGTAGTCTCTGTGAACATTCGCTAGCACACGTGTGATCAGATATCatGGCtGCTCTtA
GACCACCATGGGATGGTCATGTATCATCCTTTTTCTAGTAGCAACTGCAACCGGTGTACATTCATACAT
CCACGTGACCCAGTCTCCGTCCTCCCTGTCTGTGTCTATTGGAGACAGAGTCACCATCaacTGCCAGACG
AGTCAGGGTGTTGGCAGTGACCTACATTGGTATCAACACAAACCGGGGAGAGCCCCTAAACTCTTGAT
CCACCATACCTCTTCTGTGGAAGACGGTGTCCCCTCAAGATTCAGCGGCTCTGGATTTCACACATCTTT
TAATCTGACCATCAGCGACCTACAGGCTGACGACATTGCCACATATTACTGTCAAGTTTTACAAttctttGG
CCGAGGGAGTagaCTCCATATTAAACGTACGGTGGCTGCACCATCTGTCTTCATCTTCccaCCATCTGATG
AGCAGTTGAAATCTGGAACTGCCTCTGTTGTGTGCCTGCTGAATAACTTCTACCCCAGAGAAGCCAAA
GTGCAGTGGAAGGTGGACaatGCCctcCAGAGCGGAAACAGCCAGGAAtctGTGACAGAGCAGGATTCCAA
GGATTCCACATACAGCCTGAGCAGCACACTGACACTGTCCAAGgctGACTACGAGAAGCACAAGGTGTA
CGCCTGCGAAGTGACACACCAGGGACTGTCCTCCCCTGTGACAAAGAGCTTCAACAGAGGAGAATGCT
GATAGGATaagctttGATCTAAATAAACTTATAAATTGTGAGAGAAATTAATGAATGTCTAAGTTAATGCA
GAAACGGAGAGACATACTATATTCATGAACTAAAAGACTTAATATTGTGAAGGTATACTTTCTTTCCAC
ATAAATTTGTAGTCAATATGTTCACCCCAAAAAAGCTGTTTGTTAACTTGCCAACCTCATTCTAAAATG
TATATAGAAGCCCAAAAGACAATAACAAAAATATTCTTGTAGAACAAAATGGGAAAGAATGTTCCACT
AAATATCAAGATTTAGAGCAAAGCATGAGATGTGTGGGGATAGACAGTGAGGCTGATAAAATAGAGT
AGAGCTCAGAAACAGACCCATTGATATATGTAAGTGACCTATGAAAAAAATATGGCATTTTACAATGG
GAAAATGATGATCTTTTTCTTTTTTAGAAAAACAGGGAAATATATTTATATGTAAAAAATAAAAGGGA
ACCCATATGTCATACCATACACACAAAAAAATTCCAGTGAATTATAAGTCTAAATGGAGAAGGCAAAA
CTTTAAATCTTTTAGAAAATAATATAGAAGCATGCCATCATGACTTCAGTGTAGAGAAAAATTTCTTAT
GACTCAAAGTCCTAACCACAAAGAAAAGATTGTTAATTAGATTGCATGAATATTAAGACTTATTTTTAA
AATTAAAAAACCATTAAGAAAAGTCAGGCCATAGAATGACAGAAAATATTTGCAACACCCCAGTAAA
GAGAATTGTAATATGCAGATTATAAAAAGAAGTCTTACAAATCAGTAAAAAATAAAACTAGACAAAA
ATTTGAACAGATGAAAGAGAAACTCTAAATAATCATTACACATGAGAAACTCAATCTCAGAAATCAGA
GAACTATCATTGCATATACACTAAATTAGAGAAATATTAAAAGGCTAAGTAACATCTGTGGCAATATT
GATGGTATATAACCTTGATATGATGTGATGAGAACAGTACTTTACCCCATGGGCTTCCTCCCCAAACCC
TTACCCCAGTATAAATCATGACAAATATACTTTAAAAACCATTACCCTATATCTAACCAGTACTCCTCA
AAACTGTCAAGGTCATCAAAAATAAGAAAAGTCTGAGGAACTGTCAAAACTAAGAGGAACCCAAGGA
GACATGAGAATTATATGTAATGTGGCATTCTGAATGAGATCCCAGAACAGAAAAAGAACAGTAGCTA
AAAAACTAATGAAATATAAATAAAGTTTGAACTTTAGTTTTTTTTAAAAAAGAGTAGCATTAACACGG
CAAAGCCATTTTCATATTTTTCTTGAACATTAAGTACAAGTCTATAATTAAAAATTTTTTAAATGTAGTC
TGGAACATTGCCAGAAACAGAAGTACAGCAGCTATCTGTGCTGTCGCCTAACTATCCATAGCTGATTG
GTCTAAAATGAGATACATCAACGCTCCTCCATGTTTTTTGTTTTCTTTTTAAATGAAAAACTTTATTTTT
TAAGAGGAGTTTCAGGTTCATAGCAAAATTGAGAGGAAGGTACATTCAAGCTGAGGAAGTTTTCCTCT
ATTCCTAGTTTACTGAGAGATTGCATCATGAATGGGTGTTAAATTTTGTCAAATGCTTTTTCTGTGTCTA
TCAATATGACCATGTGATTTTCTTCTTTAACCTGTTGATGGGACAAATTACGTTAATTGATTTTCAAACG
TTGAACCACCCTTACATATCTGGAATAAATTCTACTTGGTTGTGGTGTATATTTTTTGATACATTCTTGG
ATTCTTTTTGCTAATATTTTGTTGAAAATGTTTGTATCTTTGTTCATGAGAGATATTGGTCTGTTGTTTTC
TTTTCTTGTAATGTCATTTTCTAGTTCCGGTATTAAGGTAATGCTGGCCTAGTTGAATGATTTAGGAAGT
ATTCCCTCTGCTTCTGTCTTCTGAAAGAGATTGTAGAAAGTTGATACAATTTTTTTTTCTTTAAATATCT
TGATAGAATTggatcctatcaatcGTCGACtGTACCttGGGCCtGGGACCGGAACTGAAACATAAAATGAATG
CAAcTGTTGTTGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCATGT
CTGGATCACGCGTAGtctagataccgttcgtatagcatacattatacgaagttatgtttcggccggtggatcca
ccggtattgggatcttcacacagcaggtaaggttgcgggccgggcctgggccgggtccgggccgggtattgccc
gcctaatgagcgggcttttttttcttaccccttcttccgcttcctcgctcactgactcgctgcgctcggtcgtt
cggctgcggcgagcggtatcagctcactcaaaggcggtaatacggttatccacagaatcaggggataacgcagg
aaagaacatgtgagcaaaaggccagcaaaaggccaggaaccgtaaaaaggccgcgttgctggcgtttttccata
ggctccgcccccctgacgagcatcacaaaaatcgacgctcaagtcagaggtggcgaaacccgacaggactataa
agataccaggcgtttccccctggaagctccctcgtgcgctctcctgttccgaccctgccgcttaccggatacct
gtccgcctttctcccttcgggaagcgtggcgctttctcatagctcacgctgtaggtatctcagttcggtgtagg
tcgttcgctccaagctgggctgtgtgcacgaaccccccgttcagcccgaccgctgcgccttatccggtaactat
cgtcttgagtccaacccggtaagacacgacttatcgccactggcagcagccactggtaacaggattagcagagc
gaggtatgtaggcggtgctacagagttcttgaagtggtggcctaactacggctacactagaagaacagtatttg
gtatctgcgctctgctgaagccagttaccttcggaaaaagagttggtagctcttgatccggcaaacaaaccacc
aatcattaggtaccccggcccgcactgacccctggtgttgctttttttttttaggccgcaagctgaagc
gtgtcgctggtagcggtggtttttttgtttgcaagcagcagattacgcgcagaaaaaaaggatctcaagaa
gatcctttgatcttttctacggggtctgacgctcagtggaacgaaaactcacgttaagggattttggt
catgagattatcaaaaaggatcttcacctagatccttttaaattaaaaatgaagttttaaatcaatctaa
agtatatatgagtaaacttggtctgacagttaccaatgcttaatcagtgaggcacctatctcagcga
tctgtctatttcgttcatccatagttgcctgactcctgcaaaccacgttgtggtagaattggtaaagaga
gtcgtgtaaaatatcgagttcgcacatcttgttgtctgattattgatttttggcgaaaccatttgatcatatga
caagatgtgtatctaccttaacttaatgattttgataaacactagctgactagctaaccgcggaaacagTTACC
AATGCTTAATCAGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCCAACTGATCTTCA
GCATCTTTTACTTTCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGC
GCACATTTCCCCGAAAATTTCCCCGAAAAGTGCCACCTGACGTCTAAGAAACCATTATTATCATGACAT
TAACCTATAAAAATAGGCGTATCACGAGGCCCTTTCGTC
SEQ ID NO: 3
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTCGGTAC
CTCGCGAATGCATCTAGAgcggccgcataacttcgtatagcatacattatacgaacggtaACCTGGGTACCCAT
AGAGCCCACCGCATCCCCAGCATGCCTGCTATTGTCTTCCCAATCCTCCCCCTTGCTGTCCTGCCCCACCCCAC
CCCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCATTTACCtggAGACAGGGAGAGGCTCTTCTGCGTGTAGTGG
TTGTGCAGAGCCTCATGCATCACGGAGCATGAGAAGACGTTCCCCTGCTGCCACCTGCTCTTGTCCACG
GTGAGCTTGCTGTAGAGGAAGAAGGAGCCatcagaGTCCAGCACGGGAGGCGTGGTCTTGTAGTTGTTCT
CCGGCTGCCCATTGCTCTCCCACTCCACagcGATGTCGCTGGGATAGAAGCCTTTGACCAGGCAGGTCA
GGCTGACCTGGTTCTTGGTCAGCTCATCCCGGGATGGGGGCAGGGTGTACACCTGTGGTTCTCGGGGCT
GCCCTTTGGCTTTGGAGATGGTTTTCTCGATGGGGGCTGGGAGGGCTTTGTTgctGACCTTGCACTTGTA
CTCCTTGCCATTCAGCCAGTCCTGGTGCAGGACGGTGAGGACactGACCACACGGTACGTGCTGTTGTAC
TGCTCCTCcctCGGCTTTGTCTTGGCATTATGCACCTCCACtccatcCACGTACCAGTTGAACTTGACCTCAG
GGTCTTCatgGCTCACatcCACCACCACGCATGTGACCTCAGGGGTCCGGGAGATCATGAGGGTGTCCTTG
GGTTTTGGGGGGAAGAGGAAGACTGACGGTCCCCCCAGGAGTTCAGGTGCTGGGCACGGTGGGCATG
TGTGAGTTTTGTCACAAGATTTGGGCTCAACTTTCTTGTCCACCTTGGTGTTGCTGGGCTTGTGATTCAC
GTTGCAGATGTAGGTCTGGGTGCCCAAGCTGCTGGAGGGCACGGTCACCACGCTGCTGAGGGAGTAGA
GTCCTGAGGACTGTAGGACAGCCGGGAAGGTGTGCACGCCGCTGGTCAGGGCtccTGAGTTCCACGACA
CagtCACCGGTTCGGGGAAGTAGTCCTTGACCAGGCAGCCCAGGGCCGCTGTGCCCCCAGAGGTGCTCT
TGGAGGAGGGTGCCAGGGGGAAGACactTGGGCCCTTGGTactCGCCGCagaGACGACGACagtCGTTCCCT
GTCCCCAGGCATCTAAGGCCCAAGAGGAGTCtccATAGGAcctatcTCTCGCACAGTAATAGACagcCGTGTC
atcAGGTTTAAGGCCTCTGATGTCCATGTACGCAATCTCTCTATATACatcTCGtgtCAATGTGACCCTGTCC
CGAAAACCACCACCAAAATTCACagcCCCATATTGTGGCTTGATCCACCCCACCCACTCAAGTCCTCGCC
CaggGGCCTGTCGGAACCAAAATAATATGTGGGCGGTAAAGGTGTATCCAGAGGTCTGGCAGGAGACT
CTTACTGAGGCtccCGGTTTCTTCATCGCAGTCCCTGATTGTACCAGGTGCGCTCGAGAATGTACACCGG
TTGCAGTTGCTACTAGAAAAAGGATGATACATGACCATCCCATGGTGGTCTAGtGCaGttaCGATATCTGA
TCACACGTGTCGAGGACaGTGACTGCAGATTGCTTTGAATTAGCGGTGGTTTTCACAACACCTAAAAAA
GGGTTTAAAAGATACCTTTGAACCGCTAAGAAGCCCGAGAATTAGCTCCGCTCAAAACTCAAGGGGAC
AAATTCCAAAAATGACTTCCAGCGCCAGGCTGGCCTGACTAGTCTCCACCCACCAAATGTGAACAAAC
TCCAACGCCATTACATCCCCTCCCCCCGCCGCGACTAGCCGTGCTCAAAAGCCCGAGGTGACTATTGCG
GCCGATAGGACCACGGGGTCACAGGAAGCAGCAGCCGGTGAGGGACCAGGCCCTCTTCCTTTGTGTGG
GTGACTCACCCGCCCGCTCCACCGGGCTGCCGCGTCCTCCATTTTGAGCTCCTTGCAACAGGGCCCGGG
AGCGGCCATCTTTCCACGCACGCAACTGGTGCCGGACGGGATGGCCTCACCCTAGTTAGGGAGGCAGG
GCAACGCGGCGCCGCCAAGCCAGATCGTGCCGGGTGCTGGGGCCACATGGCCTCGGCACGCTAACCCC
AGCCTGGTTGCTTCGGGAAAAACCCCAGGCCTCGCCCCATCCAGGTGGCGTCGGACATGTGCTCCGAA
GGCGGGCGGGCCCCAGCCGCCACTCCTGTCCCTCCATTCCTCCCCAACCATGACCTCTCCGGGCTCCGG
GCGAGCAAGCCCCGCACCCTCCCTTTGTTAGCCCCTATTGCTGAACGGCAATCGAAGGCAGCAGGGCA
ACAACAACAAAAAAAAAAAAGACCAGAGTGCGGCCGGAGTAGCACGCGGCGGCGGCGCGGACACCA
CGCTAGGCCTCAAGCCGGACACGAGGCGAGGCTACGGGGTTGCCGCTAGGCCTCGCACTCTGCCTCCC
GCGCCGCCCGCAACTCGAAGCGGGAATGCTCGCAGCTAATCCCCGCCGACGACAGCGGGGCCCGGCC
CGCTCGGAGCAGGACCTCCAGCTCGGCGGCCCGCGGAAGCCACACCCGCCCCTCACCTGCGTTCTGAC
GGCAAACCCGTTGCGAAAAAGAACGTCCAAGGCGACTGCCGCACTTATATACCGTTCTCCCCCACCCT
CGGGAAGAGGGCGGAGCCAGCACACGACACCGCTTTCCCAGTTTGCCCCGCGCCAGCTCCCCGGTCCT
CCCCCCCACGACCAGGGTCTATGAGCTTGTGTGCTCTGCTCTCCCCGCGCTCAGCCCTGAGCGCATGCT
CCTCGCGCTGTCGTGGGGCGGCTACAGGGATTTGGGTCTGTGAGAATTTAGGGACGGTCCCTGGGCTC
TCCCCTTGCGAAGGCGATCCCTCCTTTTGTATGAATTACTCTCGGCTCCACTAGTGGGCCCTGCAaGgaatt
ctGTTAGGGTGTGGAAAGTCCCCAGGCTCCCCAGCAGGCAGAAGTATGCAAAGCATGCATCTCAATTAG
TCAGCAACCAGGTGTGGAAAGTCCCCAGGCTCCCCAGCAGGCAGAAGTATGCAAAGCATGCATCTCAA
TTAGTCAGCAACCATAGTCCcctaggGAGACATGTTTACATAACTTACGGTAAATGGCCCGCCTGGCTGAC
CGCCCAACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGTACAT
GACCTTATGGGACTTTCCTACTTGGCAGTACATCTACGTATTAGTCATCGCTATTACCATGAATTGGTTT
GATCTGATTATAACTAGTGGAGAAGAGCATGCTTGAGGGCTGAGTGCCCCTCAGTGGGCAGAGAGCAC
ATGGCCCACAGTCCCTGAGAAGTTGGGGGGAGGGGTGGGCAATTGAACTGGTGCCTAGAGAAGGTGG
GGCTTGGGTAAACTGGGAAAGTGATGTGGTGTACTGGCTCCACCTTTTTCCCCAGGGTGGGGGAGAAC
CATATATAAGTGCAGTAGTCTCTGTGAACATTCGCTAGCACACGTGTGATCAGATATCatGGCtGCTCTtA
GACCACCATGGGATGGTCATGTATCATCCTTTTTCTAGTAGCAACTGCAACCGGTGTACATTCATACAT
CCACGTGACCCAGTCTCCGTCCTCCCTGTCTGTGTCTATTGGAGACAGAGTCACCATCaacTGCCAGACG
AGTCAGGGTGTTGGCAGTGACCTACATTGGTATCAACACAAACCGGGGAGAGCCCCTAAACTCTTGAT
CCACCATACCTCTTCTGTGGAAGACGGTGTCCCCTCAAGATTCAGCGGCTCTGGATTTCACACATCTTT
TAATCTGACCATCAGCGACCTACAGGCTGACGACATTGCCACATATTACTGTCAAGTTTTACAAttctttGG
CCGAGGGAGTagaCTCCATATTAAACGTACGGTGGCTGCACCATCTGTCTTCATCTTCccaCCATCTGATG
AGCAGTTGAAATCTGGAACTGCCTCTGTTGTGTGCCTGCTGAATAACTTCTACCCCAGAGAAGCCAAA
GTGCAGTGGAAGGTGGACaatGCCctcCAGAGCGGAAACAGCCAGGAAtctGTGACAGAGCAGGATTCCAA
GGATTCCACATACAGCCTGAGCAGCACACTGACACTGTCCAAGgctGACTACGAGAAGCACAAGGTGTA
CGCCTGCGAAGTGACACACCAGGGACTGTCCTCCCCTGTGACAAAGAGCTTCAACAGAGGAGAATGCT
GATAGGATaagctttGATCTAAATAAACTTATAAATTGTGAGAGAAATTAATGAATGTCTAAGTTAATGCA
GAAACGGAGAGACATACTATATTCATGAACTAAAAGACTTAATATTGTGAAGGTATACTTTCTTTCCAC
ATAAATTTGTAGTCAATATGTTCACCCCAAAAAAGCTGTTTGTTAACTTGCCAACCTCATTCTAAAATG
TATATAGAAGCCCAAAAGACAATAACAAAAATATTCTTGTAGAACAAAATGGGAAAGAATGTTCCACT
AAATATCAAGATTTAGAGCAAAGCATGAGATGTGTGGGGATAGACAGTGAGGCTGATAAAATAGAGT
AGAGCTCAGAAACAGACCCATTGATATATGTAAGTGACCTATGAAAAAAATATGGCATTTTACAATGG
GAAAATGATGATCTTTTTCTTTTTTAGAAAAACAGGGAAATATATTTATATGTAAAAAATAAAAGGGA
ACCCATATGTCATACCATACACACAAAAAAATTCCAGTGAATTATAAGTCTAAATGGAGAAGGCAAAA
CTTTAAATCTTTTAGAAAATAATATAGAAGCATGCCATCATGACTTCAGTGTAGAGAAAAATTTCTTAT
GACTCAAAGTCCTAACCACAAAGAAAAGATTGTTAATTAGATTGCATGAATATTAAGACTTATTTTTAA
AATTAAAAAACCATTAAGAAAAGTCAGGCCATAGAATGACAGAAAATATTTGCAACACCCCAGTAAA
GAGAATTGTAATATGCAGATTATAAAAAGAAGTCTTACAAATCAGTAAAAAATAAAACTAGACAAAA
ATTTGAACAGATGAAAGAGAAACTCTAAATAATCATTACACATGAGAAACTCAATCTCAGAAATCAGA
GAACTATCATTGCATATACACTAAATTAGAGAAATATTAAAAGGCTAAGTAACATCTGTGGCAATATT
GATGGTATATAACCTTGATATGATGTGATGAGAACAGTACTTTACCCCATGGGCTTCCTCCCCAAACCC
TTACCCCAGTATAAATCATGACAAATATACTTTAAAAACCATTACCCTATATCTAACCAGTACTCCTCA
AAACTGTCAAGGTCATCAAAAATAAGAAAAGTCTGAGGAACTGTCAAAACTAAGAGGAACCCAAGGA
GACATGAGAATTATATGTAATGTGGCATTCTGAATGAGATCCCAGAACAGAAAAAGAACAGTAGCTA
AAAAACTAATGAAATATAAATAAAGTTTGAACTTTAGTTTTTTTTAAAAAAGAGTAGCATTAACACGG
CAAAGCCATTTTCATATTTTTCTTGAACATTAAGTACAAGTCTATAATTAAAAATTTTTTAAATGTAGTC
TGGAACATTGCCAGAAACAGAAGTACAGCAGCTATCTGTGCTGTCGCCTAACTATCCATAGCTGATTG
GTCTAAAATGAGATACATCAACGCTCCTCCATGTTTTTTGTTTTCTTTTTAAATGAAAAACTTTATTTTT
TAAGAGGAGTTTCAGGTTCATAGCAAAATTGAGAGGAAGGTACATTCAAGCTGAGGAAGTTTTCCTCT
ATTCCTAGTTTACTGAGAGATTGCATCATGAATGGGTGTTAAATTTTGTCAAATGCTTTTTCTGTGTCTA
TCAATATGACCATGTGATTTTCTTCTTTAACCTGTTGATGGGACAAATTACGTTAATTGATTTTCAAACG
TTGAACCACCCTTACATATCTGGAATAAATTCTACTTGGTTGTGGTGTATATTTTTTGATACATTCTTGG
ATTCTTTTTGCTAATATTTTGTTGAAAATGTTTGTATCTTTGTTCATGAGAGATATTGGTCTGTTGTTTTC
TTTTCTTGTAATGTCATTTTCTAGTTCCGGTATTAAGGTAATGCTGGCCTAGTTGAATGATTTAGGAAGT
ATTCCCTCTGCTTCTGTCTTCTGAAAGAGATTGTAGAAAGTTGATACAATTTTTTTTTCTTTAAATATCT
TGATAGAATTggatcctatcaatcGTCGACtGTACCttGGGCCtGGGACCGGAACTGAAACATAAAATGAATG
CAAcTGTTGTTGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCATGT
CTGGATCACGCGTAGtctagataccgttcgtatagcatacattatacgaagttatgtttcggccggtggatcca
ccggtattgggatcttcacacagcaggtaaggttgcgggccgggcctgggccgggtccgggccgggtattgccc
gcctaatgagcgggcttttttttcttaccccttcttccgcttcctcgctcactgactcgctgcgctcggtcgtt
cggctgcggcgagcggtatcagctcactcaaaggggtaatacggttatccacagaatcaggggataacgcagga
aagaacatgtgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacaggactataaa
gataccaggcgtttccccctggaagctccctcgtgcgctctcctgttccgaccctgccgcttaccggatacctg
tccgcctttctcccttcgggaagcgtggcgctttctcatagctcacgctgtaggtatctcagttcggtgtaggt
cgttcgctccaagctgggctgtgtgcacgaaccccccgttcagcccgaccgctgcgccttatccggtaactatc
gtcttgagtccaacccggtaagacacgacttatcgccactggcagcagccactggtaacaggattagcagagcg
aggtatgtaggcggtgctacagagttcttgaagtggtggcctaactacggctacactagaagaacagtatttgg
tatctgcgctctgctgaagccagttaccttcggaaaaagagttggtagctcttgatccggcaaacaaaccaccg
ctggtagcggtggtttttttgtttgcaagcagcagattacgcgcagaaaaaaaggatctcaagaagatc
ctttgatcttttctacggggtctgacgctcagtggaacgaaaactcacgttaagggattttggtcatgaga
ttatcaaaaaggatcttcacctagatccttttaaattaaaaatgaagttttaaatcaatctaaagta
tatatgagtaaacttggtctgacagttaccaatgcttaatcagtgaggcacctatctcagcgatctgtct
atttcgttcatccatagttgcctgactcctgcaaaccacgttgtggtagaattggtaaagagagtcgtgt
aaaatatcgagttcgcacatcttgttgtctgattattgatttttggcgaaaccatttgatcatatgacaag
atgtgtatctaccttaacttaatgattttgataaaaatcattaggtaccccggcccgcactgacccctggt
gttgctttttttttttaggccgcaagctgaagcgtgtccactagctgactagctaaccgcggaaacagTTACC
AATGCTTAATCAGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCCAACTGATCTTCA
GCATCTTTTACTTTCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGC
GCACATTTCCCCGAAAATTTCCCCGAAAtccctatcagtgatagagaaaagtgaaagtcgagtttaccactccc
tatcagtgatagagaaaagtgaaagtcgagtttaccactccctatcagtgatagagaaaagtgaaagtcgagtt
taccactccctatcagtgatagagaaaagtgaaagtcgagtttaccactccctatcagtgatagagaaaagtga
aagtcgagtttaccactccctatcagtgatagagaaaagtgaaagtcgagtttaccactccctatcagtgatag
agaAGTGCCACCTGACGTCTAAGAAACCATTATTATCATGACATTAACCTATAAAAATAGGCGTATCACGAGGC
CCTTTCGTC
SEQ ID NO: 4
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTC
GGTACCTCGCGAATGCATCTAGAgcggccgcataacttcgtatagcatacattatacgaacggtaACCTGGGTA
CCCATAGAGCCCACCGCATCCCCAGCATGCCTGCTATTGTCTTCCCAATCCTCCCCCTTGCTGTCCTGCCCCAC
CCCACCCCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCAcaggcgcagctggtgcagagcggcccggaagtgcgcaaaccgggc
accagcgtgaaagtgagctgcaaagcgccgggcaacaccctgaaaacctatgatctgcattgggtgcgcagcgt
gccgggccagggcctgcagtggatgggctggattagccatgaaggcgataaaaaagtgattgtggaacgcttta
aagcgaaagtgaccattgattgggatcgcagcaccaacaccgcgtatctgcagctgagcggcctgaccagcggc
gataccgcggtgtattattgcgcgaaaggcagcaaacatcgcctgcgcgattatgcgctgnnngatgatgatgg
cgcgctgaactgggcggtggatgtggattatctgagcaacctggaattttggggccagggcaccgcggtgaccg
tgagcagcgcgagcaccaaaggcccgagcgtgtttccgctggcgccgagcagcaaaagcaccagcggcggcacc
gcggcgctgggctgcctggtgaaagattattttccggaaccggtgaccgtgagctggaacagcggcgcgctgac
cagcggcgtgcatacctttccggcggtgctgcagagcagcggcctgtatagcctgagcagcgtggtgaccgtgc
cgagcagcagcctgggcacccagacctatatttgcaacgtgaaccataaaccgagcaacaccaaagtggataaa
aaagtggaaccgaaaagctgcgatAGAATGTACACCGGTTGCAGTTGCTACTAGAAAAAGGATGATACATGA
CCATCCCATGGTGGTCTAGtGCaGttaCGATATCTGATCACACGTGTCGAGGACaGTGACTGCAGATTGCT
TTGAATTAGCGGTGGTTTTCACAACACCTAAAAAAGGGTTTAAAAGATACCTTTGAACCGCTAAGAAG
CCCGAGAATTAGCTCCGCTCAAAACTCAAGGGGACAAATTCCAAAAATGACTTCCAGCGCCAGGCTGG
CCTGACTAGTCTCCACCCACCAAATGTGAACAAACTCCAACGCCATTACATCCCCTCCCCCCGCCGCGA
CTAGCCGTGCTCAAAAGCCCGAGGTGACTATTGCGGCCGATAGGACCACGGGGTCACAGGAAGCAGC
AGCCGGTGAGGGACCAGGCCCTCTTCCTTTGTGTGGGTGACTCACCCGCCCGCTCCACCGGGCTGCCGC
GTCCTCCATTTTGAGCTCCTTGCAACAGGGCCCGGGAGCGGCCATCTTTCCACGCACGCAACTGGTGCC
GGACGGGATGGCCTCACCCTAGTTAGGGAGGCAGGGCAACGCGGCGCCGCCAAGCCAGATCGTGCCG
GGTGCTGGGGCCACATGGCCTCGGCACGCTAACCCCAGCCTGGTTGCTTCGGGAAAAACCCCAGGCCT
CGCCCCATCCAGGTGGCGTCGGACATGTGCTCCGAAGGCGGGCGGGCCCCAGCCGCCACTCCTGTCCC
TCCATTCCTCCCCAACCATGACCTCTCCGGGCTCCGGGCGAGCAAGCCCCGCACCCTCCCTTTGTTAGC
CCCTATTGCTGAACGGCAATCGAAGGCAGCAGGGCAACAACAACAAAAAAAAAAAAGACCAGAGTGC
GGCCGGAGTAGCACGCGGCGGCGGCGCGGACACCACGCTAGGCCTCAAGCCGGACACGAGGCGAGGC
TACGGGGTTGCCGCTAGGCCTCGCACTCTGCCTCCCGCGCCGCCCGCAACTCGAAGCGGGAATGCTCG
CAGCTAATCCCCGCCGACGACAGCGGGGCCCGGCCCGCTCGGAGCAGGACCTCCAGCTCGGCGGCCCG
CGGAAGCCACACCCGCCCCTCACCTGCGTTCTGACGGCAAACCCGTTGCGAAAAAGAACGTCCAAGGC
GACTGCCGCACTTATATACCGTTCTCCCCCACCCTCGGGAAGAGGGCGGAGCCAGCACACGACACCGC
TTTCCCAGTTTGCCCCGCGCCAGCTCCCCGGTCCTCCCCCCCACGACCAGGGTCTATGAGCTTGTGTGC
TCTGCTCTCCCCGCGCTCAGCCCTGAGCGCATGCTCCTCGCGCTGTCGTGGGGCGGCTACAGGGATTTG
GGTCTGTGAGAATTTAGGGACGGTCCCTGGGCTCTCCCCTTGCGAAGGCGATCCCTCCTTTTGTATGAA
TTACTCTCGGCTCCACTAGTGGGCCCTGCAaGgaattctGTTAGGGTGTGGAAAGTCCCCAGGCTCCCCAGC
AGGCAGAAGTATGCAAAGCATGCATCTCAATTAGTCAGCAACCAGGTGTGGAAAGTCCCCAGGCTCCC
CAGCAGGCAGAAGTATGCAAAGCATGCATCTCAATTAGTCAGCAACCATAGTCCcctaggGAGACATGTT
TACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCAGTACATC
TACGTATTAGTCATCGCTATTACCATGAATTGGTTTGATCTGATTATAACTAGTGGAGAAGAGCATGCT
TGAGGGCTGAGTGCCCCTCAGTGGGCAGAGAGCACATGGCCCACAGTCCCTGAGAAGTTGGGGGGAG
GGGTGGGCAATTGAACTGGTGCCTAGAGAAGGTGGGGCTTGGGTAAACTGGGAAAGTGATGTGGTGT
ACTGGCTCCACCTTTTTCCCCAGGGTGGGGGAGAACCATATATAAGTGCAGTAGTCTCTGTGAACATTC
GCTAGCACACGTGTGATCAGATATCatGGCtGCTCTtAGACCACCATGGGATGGTCATGTATCATCCTTTT
TCTAGTAGCAACTGCAACCGGTGTACATTCAgattttgtgctgacccagagcccgcatagcctgagcgtgaccc
cgggcgaaagcgcgagcattagctgcaaaagcagccatagcctgattcatggcgatcgcaacaactatctggcg
tggtatgtgcagaaaccgggccgcagcccgcagctgctgatttatctggcgagcagccgcgcgagcggcgtgcc
ggatcgctttagcggcagcggcagcgataaagattttaccctgaaaattagccgcgtggaaaccgaagatgtgg
gcacctattattgcatgcagggccgcgaaagcccgtggacctttggccagggcaccaaagtggatattaaacgc
accgtggggcgccgagcgtgtttatttttccgccgagcgatgaacagctgaaaagcggcaccgcgagcgtggtg
tgcctgctgaacaacttttatccgcgcgaagcgaaagtgcagtggaaagtggataacgcgctgcagag
cggcaacagccaggaaagcgtgaccgaacaggatagcaaagatagcacctatagcctgagcagcaccctg
accctgagcaaagcggattatgaaaaacataaagtgtatgcgtgcgaagtgacccatcagggcctgagcag
cccggtgaccaaaagctttaaccgcggcgaatgcTGATAGGATaagctttGATCTAAATAAACTTATAAA
TTGTGAGAGAAATTAATGAATGTCTAAGTTAATGCAGAAACGGAGAGACATACTATATTCATGAACTA
AAAGACTTAATATTGTGAAGGTATACTTTCTTTCCACATAAATTTGTAGTCAATATGTTCACCCCAAAA
AAGCTGTTTGTTAACTTGCCAACCTCATTCTAAAATGTATATAGAAGCCCAAAAGACAATAACAAAAA
TATTCTTGTAGAACAAAATGGGAAAGAATGTTCCACTAAATATCAAGATTTAGAGCAAAGCATGAGAT
GTGTGGGGATAGACAGTGAGGCTGATAAAATAGAGTAGAGCTCAGAAACAGACCCATTGATATATGT
AAGTGACCTATGAAAAAAATATGGCATTTTACAATGGGAAAATGATGATCTTTTTCTTTTTTAGAAAAA
CAGGGAAATATATTTATATGTAAAAAATAAAAGGGAACCCATATGTCATACCATACACACAAAAAAAT
TCCAGTGAATTATAAGTCTAAATGGAGAAGGCAAAACTTTAAATCTTTTAGAAAATAATATAGAAGCA
TGCCATCATGACTTCAGTGTAGAGAAAAATTTCTTATGACTCAAAGTCCTAACCACAAAGAAAAGATT
GTTAATTAGATTGCATGAATATTAAGACTTATTTTTAAAATTAAAAAACCATTAAGAAAAGTCAGGCC
ATAGAATGACAGAAAATATTTGCAACACCCCAGTAAAGAGAATTGTAATATGCAGATTATAAAAAGA
AGTCTTACAAATCAGTAAAAAATAAAACTAGACAAAAATTTGAACAGATGAAAGAGAAACTCTAAAT
AATCATTACACATGAGAAACTCAATCTCAGAAATCAGAGAACTATCATTGCATATACACTAAATTAGA
GAAATATTAAAAGGCTAAGTAACATCTGTGGCAATATTGATGGTATATAACCTTGATATGATGTGATG
AGAACAGTACTTTACCCCATGGGCTTCCTCCCCAAACCCTTACCCCAGTATAAATCATGACAAATATAC
TTTAAAAACCATTACCCTATATCTAACCAGTACTCCTCAAAACTGTCAAGGTCATCAAAAATAAGAAA
AGTCTGAGGAACTGTCAAAACTAAGAGGAACCCAAGGAGACATGAGAATTATATGTAATGTGGCATTC
TGAATGAGATCCCAGAACAGAAAAAGAACAGTAGCTAAAAAACTAATGAAATATAAATAAAGTTTGA
ACTTTAGTTTTTTTTAAAAAAGAGTAGCATTAACACGGCAAAGCCATTTTCATATTTTTCTTGAACATTA
AGTACAAGTCTATAATTAAAAATTTTTTAAATGTAGTCTGGAACATTGCCAGAAACAGAAGTACAGCA
GCTATCTGTGCTGTCGCCTAACTATCCATAGCTGATTGGTCTAAAATGAGATACATCAACGCTCCTCCA
TGTTTTTTGTTTTCTTTTTAAATGAAAAACTTTATTTTTTAAGAGGAGTTTCAGGTTCATAGCAAAATTG
AGAGGAAGGTACATTCAAGCTGAGGAAGTTTTCCTCTATTCCTAGTTTACTGAGAGATTGCATCATGAA
TGGGTGTTAAATTTTGTCAAATGCTTTTTCTGTGTCTATCAATATGACCATGTGATTTTCTTCTTTAACCT
GTTGATGGGACAAATTACGTTAATTGATTTTCAAACGTTGAACCACCCTTACATATCTGGAATAAATTC
TACTTGGTTGTGGTGTATATTTTTTGATACATTCTTGGATTCTTTTTGCTAATATTTTGTTGAAAATGTTT
GTATCTTTGTTCATGAGAGATATTGGTCTGTTGTTTTCTTTTCTTGTAATGTCATTTTCTAGTTCCGGTAT
TAAGGTAATGCTGGCCTAGTTGAATGATTTAGGAAGTATTCCCTCTGCTTCTGTCTTCTGAAAGAGATTGTA
GAAAGTTGATACAATTTTTTTTTCTTTAAATATCTTGATAGAATTggatcctatcaatcGTCGACtGTACCttG
GGCCtGGGACCGGAACTGAAACATAAAATGAATGCAAcTGTTGTTGTTAACTTGTTTATTGCAGCTTAT
AATGGTTACAAATAAAGCAATAGCATCACAAATTTCACAAATAAAGCATTTTTTTCACTGCATTCTAGTTGTGG
TTTGTCCAAACTCATCAATGTATCTTATCATGTCTGGATCACGCGTAGtctagataccgttcgtatagcataca
ttatacgaagttatgtttaaacagCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTG
CGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTCTAAGAAA
CCATTATTATCATGACATTAACCTATAAAAATAGGCGTATCACGAGGCCCTTTCGTC
SEQ ID NO: 5
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTCGGTAC
CTCGCGAATGCATCTAGAgcggccgcataacttcgtatagcatacattatacgaacggtaACCTGGGTACCCAT
AGAGCCCACCGCATCCCCAGCATGCCTGCTATTGTCTTCCCAATCCTCCCCCTTGCTGTCCTGCCCCACCCCAC
CCCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCAcaggcgcagctggtgcagagcggcccggaagtgcgcaaaccgggc
accagcgtgaaagtgagctgcaaagcgccgggcaacaccctgaaaacctatgatctgcattgggtgcgcagcgt
gccgggccagggcctgcagtggatgggctggattagccatgaaggcgataaaaaagtgattgtggaacgcttta
aagcgaaagtgaccattgattgggatcgcagcaccaacaccgcgtatctgcagctgagcggcctgaccagcggc
gataccgcggtgtattattgcgcgaaaggcagcaaacatcgcctgcgcgattatgcgctgnnngatgatgatgg
cgcgctgaactgggcggtggatgtggattatctgagcaacctggaattttggggccagggcaccgcggtgaccg
tgagcagcgcgagcaccaaaggcccgagcgtgtttccgctggcgccgagcagcaaaagcaccagcggcggcacc
gcggcgctgggctgcctggtgaaagattattttccggaaccggtgaccgtgagctggaacagcggcgcgctgac
cagcggcgtgcatacctttccggcggtgctgcagagcagcggcctgtatagcctgagcagcgtggtgaccgtgc
cgagcagcagcctgggcacccagacctatatttgcaacgtgaaccataaaccgagcaacaccaaagtggataaa
aaagtggaaccgaaaagctgcgatAGAATGTACACCGGTTGCAGTTGCTACTAGAAAAAGGATGATACATGA
CCATCCCATGGTGGTCTAGtGCaGttaCGATATCTGATCACACGTGTCGAGGACaGTGACTGCAGATTGCT
TTGAATTAGCGGTGGTTTTCACAACACCTAAAAAAGGGTTTAAAAGATACCTTTGAACCGCTAAGAAG
CCCGAGAATTAGCTCCGCTCAAAACTCAAGGGGACAAATTCCAAAAATGACTTCCAGCGCCAGGCTGG
CCTGACTAGTCTCCACCCACCAAATGTGAACAAACTCCAACGCCATTACATCCCCTCCCCCCGCCGCGA
CTAGCCGTGCTCAAAAGCCCGAGGTGACTATTGCGGCCGATAGGACCACGGGGTCACAGGAAGCAGC
AGCCGGTGAGGGACCAGGCCCTCTTCCTTTGTGTGGGTGACTCACCCGCCCGCTCCACCGGGCTGCCGC
GTCCTCCATTTTGAGCTCCTTGCAACAGGGCCCGGGAGCGGCCATCTTTCCACGCACGCAACTGGTGCC
GGACGGGATGGCCTCACCCTAGTTAGGGAGGCAGGGCAACGCGGCGCCGCCAAGCCAGATCGTGCCG
GGTGCTGGGGCCACATGGCCTCGGCACGCTAACCCCAGCCTGGTTGCTTCGGGAAAAACCCCAGGCCT
CGCCCCATCCAGGTGGCGTCGGACATGTGCTCCGAAGGCGGGCGGGCCCCAGCCGCCACTCCTGTCCC
TCCATTCCTCCCCAACCATGACCTCTCCGGGCTCCGGGCGAGCAAGCCCCGCACCCTCCCTTTGTTAGC
CCCTATTGCTGAACGGCAATCGAAGGCAGCAGGGCAACAACAACAAAAAAAAAAAAGACCAGAGTGC
GGCCGGAGTAGCACGCGGCGGCGGCGCGGACACCACGCTAGGCCTCAAGCCGGACACGAGGCGAGGC
TACGGGGTTGCCGCTAGGCCTCGCACTCTGCCTCCCGCGCCGCCCGCAACTCGAAGCGGGAATGCTCG
CAGCTAATCCCCGCCGACGACAGCGGGGCCCGGCCCGCTCGGAGCAGGACCTCCAGCTCGGCGGCCCG
CGGAAGCCACACCCGCCCCTCACCTGCGTTCTGACGGCAAACCCGTTGCGAAAAAGAACGTCCAAGGC
GACTGCCGCACTTATATACCGTTCTCCCCCACCCTCGGGAAGAGGGCGGAGCCAGCACACGACACCGC
TTTCCCAGTTTGCCCCGCGCCAGCTCCCCGGTCCTCCCCCCCACGACCAGGGTCTATGAGCTTGTGTGC
TCTGCTCTCCCCGCGCTCAGCCCTGAGCGCATGCTCCTCGCGCTGTCGTGGGGCGGCTACAGGGATTTG
GGTCTGTGAGAATTTAGGGACGGTCCCTGGGCTCTCCCCTTGCGAAGGCGATCCCTCCTTTTGTATGAA
TTACTCTCGGCTCCACTAGTGGGCCCTGCAaGgaattctGTTAGGGTGTGGAAAGTCCCCAGGCTCCCCAGC
AGGCAGAAGTATGCAAAGCATGCATCTCAATTAGTCAGCAACCAGGTGTGGAAAGTCCCCAGGCTCCC
CAGCAGGCAGAAGTATGCAAAGCATGCATCTCAATTAGTCAGCAACCATAGTCCcctaggGAGACATGTT
TACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCAGTACATC
TACGTATTAGTCATCGCTATTACCATGAATTGGTTTGATCTGATTATAACTAGTGGAGAAGAGCATGCT
TGAGGGCTGAGTGCCCCTCAGTGGGCAGAGAGCACATGGCCCACAGTCCCTGAGAAGTTGGGGGGAG
GGGTGGGCAATTGAACTGGTGCCTAGAGAAGGTGGGGCTTGGGTAAACTGGGAAAGTGATGTGGTGT
ACTGGCTCCACCTTTTTCCCCAGGGTGGGGGAGAACCATATATAAGTGCAGTAGTCTCTGTGAACATTC
GCTAGCACACGTGTGATCAGATATCatGGCtGCTCTtAGACCACCATGGGATGGTCATGTATCATCCTTTT
TCTAGTAGCAACTGCAACCGGTGTACATTCAgattttgtgctgacccagagcccgcatagcctgagcgtgaccc
cgggcgaaagcgcgagcattagctgcaaaagcagccatagcctgattcatggcgatcgcaacaactatctggcg
tggtatgtgcagaaaccgggccgcagcccgcagctgctgatttatctggcgagcagccgcgcgagcggcgtgcc
ggatcgctttagcggcagcggcagcgataaagattttaccctgaaaattagccgcgtggaaaccgaagatgtgg
gcacctattattgcatgcagggccgcgaaagcccgtggacctttggccagggcaccaaagtggatattaaacgc
accgtggcggcgccgagcgtgtttatttttccgccgagcgatgaacagctgaaaagcggcaccgcgagcgtggt
gtgcctgctgaacaacttttatccgcgcgaagcgaaagtgcagtggaaagtggataacgcgctgcagag
cggcaacagccaggaaagcgtgaccgaacaggatagcaaagatagcacctatagcctgagcagcaccc
tgaccctgagcaaagcggattatgaaaaacataaagtgtatgcgtgcgaagtgacccatcagggcctgagc
agcccggtgaccaaaagctttaaccgcggcgaatgcTGATAGGATaagctttGATCTAAATAAACTTATAAA
TTGTGAGAGAAATTAATGAATGTCTAAGTTAATGCAGAAACGGAGAGACATACTATATTCATGAACTA
AAAGACTTAATATTGTGAAGGTATACTTTCTTTCCACATAAATTTGTAGTCAATATGTTCACCCCAAAA
AAGCTGTTTGTTAACTTGCCAACCTCATTCTAAAATGTATATAGAAGCCCAAAAGACAATAACAAAAA
TATTCTTGTAGAACAAAATGGGAAAGAATGTTCCACTAAATATCAAGATTTAGAGCAAAGCATGAGAT
GTGTGGGGATAGACAGTGAGGCTGATAAAATAGAGTAGAGCTCAGAAACAGACCCATTGATATATGT
AAGTGACCTATGAAAAAAATATGGCATTTTACAATGGGAAAATGATGATCTTTTTCTTTTTTAGAAAAA
CAGGGAAATATATTTATATGTAAAAAATAAAAGGGAACCCATATGTCATACCATACACACAAAAAAAT
TCCAGTGAATTATAAGTCTAAATGGAGAAGGCAAAACTTTAAATCTTTTAGAAAATAATATAGAAGCA
TGCCATCATGACTTCAGTGTAGAGAAAAATTTCTTATGACTCAAAGTCCTAACCACAAAGAAAAGATT
GTTAATTAGATTGCATGAATATTAAGACTTATTTTTAAAATTAAAAAACCATTAAGAAAAGTCAGGCC
ATAGAATGACAGAAAATATTTGCAACACCCCAGTAAAGAGAATTGTAATATGCAGATTATAAAAAGA
AGTCTTACAAATCAGTAAAAAATAAAACTAGACAAAAATTTGAACAGATGAAAGAGAAACTCTAAAT
AATCATTACACATGAGAAACTCAATCTCAGAAATCAGAGAACTATCATTGCATATACACTAAATTAGA
GAAATATTAAAAGGCTAAGTAACATCTGTGGCAATATTGATGGTATATAACCTTGATATGATGTGATG
AGAACAGTACTTTACCCCATGGGCTTCCTCCCCAAACCCTTACCCCAGTATAAATCATGACAAATATAC
TTTAAAAACCATTACCCTATATCTAACCAGTACTCCTCAAAACTGTCAAGGTCATCAAAAATAAGAAA
AGTCTGAGGAACTGTCAAAACTAAGAGGAACCCAAGGAGACATGAGAATTATATGTAATGTGGCATTC
TGAATGAGATCCCAGAACAGAAAAAGAACAGTAGCTAAAAAACTAATGAAATATAAATAAAGTTTGA
ACTTTAGTTTTTTTTAAAAAAGAGTAGCATTAACACGGCAAAGCCATTTTCATATTTTTCTTGAACATTA
AGTACAAGTCTATAATTAAAAATTTTTTAAATGTAGTCTGGAACATTGCCAGAAACAGAAGTACAGCA
GCTATCTGTGCTGTCGCCTAACTATCCATAGCTGATTGGTCTAAAATGAGATACATCAACGCTCCTCCA
TGTTTTTTGTTTTCTTTTTAAATGAAAAACTTTATTTTTTAAGAGGAGTTTCAGGTTCATAGCAAAATTG
AGAGGAAGGTACATTCAAGCTGAGGAAGTTTTCCTCTATTCCTAGTTTACTGAGAGATTGCATCATGAA
TGGGTGTTAAATTTTGTCAAATGCTTTTTCTGTGTCTATCAATATGACCATGTGATTTTCTTCTTTAACCT
GTTGATGGGACAAATTACGTTAATTGATTTTCAAACGTTGAACCACCCTTACATATCTGGAATAAATTC
TACTTGGTTGTGGTGTATATTTTTTGATACATTCTTGGATTCTTTTTGCTAATATTTTGTTGAAAATGTTT
GTATCTTTGTTCATGAGAGATATTGGTCTGTTGTTTTCTTTTCTTGTAATGTCATTTTCTAGTTCCGGTAT
TAAGGTAATGCTGGCCTAGTTGAATGATTTAGGAAGTATTCCCTCTGCTTCTGTCTTCTGAAAGAGATTGTA
GAAAGTTGATACAATTTTTTTTTCTTTAAATATCTTGATAGAATTggatcctatcaatcGTCGACtGTACCttG
GGCCtGGGACCGGAACTGAAACATAAAATGAATGCAAcTGTTGTTGTTAACTTGTTTATTGCAGCTTAT
AATGGTTACAAATAAAGCAATAGCATCACAAATTTCACAAATAAAGCATTTTTTTCACTGCATTCTAGT
TGTGGTTTGTCCAAACTCATCAATGTATCTTATCATGTCTGGATCACGCGTAGtctagataccgttcgtatagc
atacattatacgaagttatACGCGTAGtctagagtttcggccggtggatccaccggtattgggatcttcacaca
gcaggtaaggttgcgggccgggcctgggccgggtccgggccgggtattgcccgcctaatgagcgggcttttttt
tcttaccccttcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggc
cagcaaaaggccaggaaccgtaaaaaggccgcgttgctggcgtttttccataggctccgcccccctgacgagca
tcacaaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttcggga
agcgtggcgctttctcatagctcacgctgtaggtatctcagttcggtgtaggtcgttcgctccaagctgggctg
tgtgcacgaaccccccgttcagcccgaccgctgcgccttatccggtaactatcgtcttgagtccaacccggtaa
gacacgacttatcgccactggcagcagccactggtaacaggattagcagagcgaggtatgtaggcggtgctaca
gagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggt
ctgacgctcagtggaacgaaaactcacgttaagggattttggtcatgagattatcaaaaaggatc
ttcacctagatccttttaaattaaaaatgaagttttaaatcaatctaaagtatatatgagtaaacttgg
tctgacagttaccaatgcttaatcagtgaggcacctatctcagcgatctgtctatttcgttcatccat
agttgcctgactcctgcaaaccacgttgtggtagaattggtaaagagagtcgtgtaaaatatcgagttcg
cacatcttgttgtctgattattgatttttggcgaaaccatttgatcatatgacaagatgtgtatctaccttaac
ttaatgattttgataaaaatcattaggtaccccggcccgcactgacccctggtgttgctttttttttttaggcc
gcaagctgaagcgtgtccactagctgactagctaaccgcggaaacagTTACCAATGCTTAATCAGTGAGGCAC
CTATCTCAGCGATCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCATAATTCTC
TTACTGTCATGCCATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACCGCTGTTG
AGATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAATTTCCCCG
AAAAGTGCCACCTGACGTCTAAGAAACCATTATTATCATGACATTAACCTATAAAAATAGGCGTATCA
CGAGGCCCTTTCGTC
SEQ ID NO: 6
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTCGAGCTCGGTAC
CTCGCGAATGCATCTAGAgcggccgcataacttcgtatagcatacattatacgaacggtaACCTGGGTACCCAT
AGAGCCCACCGCATCCCCAGCATGCCTGCTATTGTCTTCCCAATCCTCCCCCTTGCTGTCCTGCCCCACCCCAC
CCCCCAGAATAGAATGACACCTACTCAGACAATGCGATGCAATTTCCTCATTTTATTAGGAAAGGACAG
TGGGAGTGGCACCTTCCAGGGTCAAGGAAGGCACGGGGGAGGGGCAAACAACAGATGGCTGGCAACT
AGAAGGCACAGCAGATCTGATCCTCATCAcaggcgcagctggtgcagagcggcccggaagtgcgcaaaccgggc
accagcgtgaaagtgagctgcaaagcgccgggcaacaccctgaaaacctatgatctgcattgggtgcgcagcgt
gccgggccagggcctgcagtggatgggctggattagccatgaaggcgataaaaaagtgattgtggaacgcttta
aagcgaaagtgaccattgattgggatcgcagcaccaacaccgcgtatctgcagctgagcggcctgaccagcggc
gataccgcggtgtattattgcgcgaaaggcagcaaacatcgcctgcgcgattatgcgctgnnngatgatgatgg
cgcgctgaactgggcggtggatgtggattatctgagcaacctggaattttggggccagggcaccgcggtgaccg
tgagcagcgcgagcaccaaaggcccgagcgtgtttccgctggcgccgagcagcaaaagcaccagcggcggcacc
gcggcgctgggctgcctggtgaaagattattttccggaaccggtgaccgtgagctggaacagcggcgcgctgac
cagcggcgtgcatacctttccggcggtgctgcagagcagcggcctgtatagcctgagcagcgtggtgaccgtgc
cgagcagcagcctgggcacccagacctatatttgcaacgtgaaccataaaccgagcaacaccaaagtggataaa
aaagtggaaccgaaaagctgcgatAGAATGTACACCGGTTGCAGTTGCTACTAGAAAAAGGATGATACATGA
CCATCCCATGGTGGTCTAGtGCaGttaCGATATCTGATCACACGTGTCGAGGACaGTGACTGCAGATTGCT
TTGAATTAGCGGTGGTTTTCACAACACCTAAAAAAGGGTTTAAAAGATACCTTTGAACCGCTAAGAAG
CCCGAGAATTAGCTCCGCTCAAAACTCAAGGGGACAAATTCCAAAAATGACTTCCAGCGCCAGGCTGG
CCTGACTAGTCTCCACCCACCAAATGTGAACAAACTCCAACGCCATTACATCCCCTCCCCCCGCCGCGA
CTAGCCGTGCTCAAAAGCCCGAGGTGACTATTGCGGCCGATAGGACCACGGGGTCACAGGAAGCAGC
AGCCGGTGAGGGACCAGGCCCTCTTCCTTTGTGTGGGTGACTCACCCGCCCGCTCCACCGGGCTGCCGC
GTCCTCCATTTTGAGCTCCTTGCAACAGGGCCCGGGAGCGGCCATCTTTCCACGCACGCAACTGGTGCC
GGACGGGATGGCCTCACCCTAGTTAGGGAGGCAGGGCAACGCGGCGCCGCCAAGCCAGATCGTGCCG
GGTGCTGGGGCCACATGGCCTCGGCACGCTAACCCCAGCCTGGTTGCTTCGGGAAAAACCCCAGGCCT
CGCCCCATCCAGGTGGCGTCGGACATGTGCTCCGAAGGCGGGCGGGCCCCAGCCGCCACTCCTGTCCC
TCCATTCCTCCCCAACCATGACCTCTCCGGGCTCCGGGCGAGCAAGCCCCGCACCCTCCCTTTGTTAGC
CCCTATTGCTGAACGGCAATCGAAGGCAGCAGGGCAACAACAACAAAAAAAAAAAAGACCAGAGTGC
GGCCGGAGTAGCACGCGGCGGCGGCGCGGACACCACGCTAGGCCTCAAGCCGGACACGAGGCGAGGC
TACGGGGTTGCCGCTAGGCCTCGCACTCTGCCTCCCGCGCCGCCCGCAACTCGAAGCGGGAATGCTCG
CAGCTAATCCCCGCCGACGACAGCGGGGCCCGGCCCGCTCGGAGCAGGACCTCCAGCTCGGCGGCCCG
CGGAAGCCACACCCGCCCCTCACCTGCGTTCTGACGGCAAACCCGTTGCGAAAAAGAACGTCCAAGGC
GACTGCCGCACTTATATACCGTTCTCCCCCACCCTCGGGAAGAGGGCGGAGCCAGCACACGACACCGC
TTTCCCAGTTTGCCCCGCGCCAGCTCCCCGGTCCTCCCCCCCACGACCAGGGTCTATGAGCTTGTGTGC
TCTGCTCTCCCCGCGCTCAGCCCTGAGCGCATGCTCCTCGCGCTGTCGTGGGGCGGCTACAGGGATTTG
GGTCTGTGAGAATTTAGGGACGGTCCCTGGGCTCTCCCCTTGCGAAGGCGATCCCTCCTTTTGTATGAA
TTACTCTCGGCTCCACTAGTGGGCCCTGCAaGgaattctGTTAGGGTGTGGAAAGTCCCCAGGCTCCCCAGC
AGGCAGAAGTATGCAAAGCATGCATCTCAATTAGTCAGCAACCAGGTGTGGAAAGTCCCCAGGCTCCC
CAGCAGGCAGAAGTATGCAAAGCATGCATCTCAATTAGTCAGCAACCATAGTCCcctaggGAGACATGTT
TACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCAGTACATC
TACGTATTAGTCATCGCTATTACCATGAATTGGTTTGATCTGATTATAACTAGTGGAGAAGAGCATGCT
TGAGGGCTGAGTGCCCCTCAGTGGGCAGAGAGCACATGGCCCACAGTCCCTGAGAAGTTGGGGGGAG
GGGTGGGCAATTGAACTGGTGCCTAGAGAAGGTGGGGCTTGGGTAAACTGGGAAAGTGATGTGGTGT
ACTGGCTCCACCTTTTTCCCCAGGGTGGGGGAGAACCATATATAAGTGCAGTAGTCTCTGTGAACATTC
GCTAGCACACGTGTGATCAGATATCatGGCtGCTCTtAGACCACCATGGGATGGTCATGTATCATCCTTTT
TCTAGTAGCAACTGCAACCGGTGTACATTCAgattttgtgctgacccagagcccgcatagcctgagcgtgaccc
cgggcgaaagcgcgagcattagctgcaaaagcagccatagcctgattcatggcgatcgcaacaactatctggcg
tggtatgtgcagaaaccgggccgcagcccgcagctgctgatttatctggcgagcagccgcgcgagcggcgtgcc
ggatcgctttagcggcagcggcagcgataaagattttaccctgaaaattagccgcgtggaaaccgaagatgtgg
gcacctattattgcatgcagggccgcgaaagcccgtggacctttggccagggcaccaaagtggatattaaacgc
accgtggcggcgccgagcgtgtttatttttccgccgagcgatgaacagctgaaaagcggcaccgcgagcgtggt
gtgcctgctgaacaacttttatccgcgcgaagcgaaagtgcagtggaaagtggataacgcgctgcagagc
ggcaacagccaggaaagcgtgaccgaacaggatagcaaagatagcacctatagcctgagcagcaccctgac
cctgagcaaagcggattatgaaaaacataaagtgtatgcgtgcgaagtgacccatcagggcctgagcag
cccggtgaccaaaagctttaaccgcggcgaatgcTGATAGGATaagctttGATCTAAATAAACTTATAAA
TTGTGAGAGAAATTAATGAATGTCTAAGTTAATGCAGAAACGGAGAGACATACTATATTCATGAACTA
AAAGACTTAATATTGTGAAGGTATACTTTCTTTCCACATAAATTTGTAGTCAATATGTTCACCCCAAAA
AAGCTGTTTGTTAACTTGCCAACCTCATTCTAAAATGTATATAGAAGCCCAAAAGACAATAACAAAAA
TATTCTTGTAGAACAAAATGGGAAAGAATGTTCCACTAAATATCAAGATTTAGAGCAAAGCATGAGAT
GTGTGGGGATAGACAGTGAGGCTGATAAAATAGAGTAGAGCTCAGAAACAGACCCATTGATATATGT
AAGTGACCTATGAAAAAAATATGGCATTTTACAATGGGAAAATGATGATCTTTTTCTTTTTTAGAAAAA
CAGGGAAATATATTTATATGTAAAAAATAAAAGGGAACCCATATGTCATACCATACACACAAAAAAAT
TCCAGTGAATTATAAGTCTAAATGGAGAAGGCAAAACTTTAAATCTTTTAGAAAATAATATAGAAGCA
TGCCATCATGACTTCAGTGTAGAGAAAAATTTCTTATGACTCAAAGTCCTAACCACAAAGAAAAGATT
GTTAATTAGATTGCATGAATATTAAGACTTATTTTTAAAATTAAAAAACCATTAAGAAAAGTCAGGCC
ATAGAATGACAGAAAATATTTGCAACACCCCAGTAAAGAGAATTGTAATATGCAGATTATAAAAAGA
AGTCTTACAAATCAGTAAAAAATAAAACTAGACAAAAATTTGAACAGATGAAAGAGAAACTCTAAAT
AATCATTACACATGAGAAACTCAATCTCAGAAATCAGAGAACTATCATTGCATATACACTAAATTAGA
GAAATATTAAAAGGCTAAGTAACATCTGTGGCAATATTGATGGTATATAACCTTGATATGATGTGATG
AGAACAGTACTTTACCCCATGGGCTTCCTCCCCAAACCCTTACCCCAGTATAAATCATGACAAATATAC
TTTAAAAACCATTACCCTATATCTAACCAGTACTCCTCAAAACTGTCAAGGTCATCAAAAATAAGAAA
AGTCTGAGGAACTGTCAAAACTAAGAGGAACCCAAGGAGACATGAGAATTATATGTAATGTGGCATTC
TGAATGAGATCCCAGAACAGAAAAAGAACAGTAGCTAAAAAACTAATGAAATATAAATAAAGTTTGA
ACTTTAGTTTTTTTTAAAAAAGAGTAGCATTAACACGGCAAAGCCATTTTCATATTTTTCTTGAACATTA
AGTACAAGTCTATAATTAAAAATTTTTTAAATGTAGTCTGGAACATTGCCAGAAACAGAAGTACAGCA
GCTATCTGTGCTGTCGCCTAACTATCCATAGCTGATTGGTCTAAAATGAGATACATCAACGCTCCTCCA
TGTTTTTTGTTTTCTTTTTAAATGAAAAACTTTATTTTTTAAGAGGAGTTTCAGGTTCATAGCAAAATTG
AGAGGAAGGTACATTCAAGCTGAGGAAGTTTTCCTCTATTCCTAGTTTACTGAGAGATTGCATCATGAA
TGGGTGTTAAATTTTGTCAAATGCTTTTTCTGTGTCTATCAATATGACCATGTGATTTTCTTCTTTAACCT
GTTGATGGGACAAATTACGTTAATTGATTTTCAAACGTTGAACCACCCTTACATATCTGGAATAAATTC
TACTTGGTTGTGGTGTATATTTTTTGATACATTCTTGGATTCTTTTTGCTAATATTTTGTTGAAAATGTTT
GTATCTTTGTTCATGAGAGATATTGGTCTGTTGTTTTCTTTTCTTGTAATGTCATTTTCTAGTTCCGGTAT
TAAGGTAATGCTGGCCTAGTTGAATGATTTAGGAAGTATTCCCTCTGCTTCTGTCTTCTGAAAGAGATT
GTAGAAAGTTGATACAATTTTTTTTTCTTTAAATATCTTGATAGAATTggatcctatcaatcGTCGACtGTACC
ttGGGCCtGGGACCGGAACTGAAACATAAAATGAATGCAAcTGTTGTTGTTAACTTGTTTATTGCAGCTTAT
AATGGTTACAAATAAAGCAATAGCATCACAAATTTCACAAATAAAGCATTTTTTTCACTGCATTCTAGT
TGTGGTTTGTCCAAACTCATCAATGTATCTTATCATGTCTGGATCACGCGTAGtctagataccgttcgtatagc
atacattatacgaagttatACGCGTAGtctagagtttcggccggtggatccaccggtattgggatcttcacaca
gcaggtaaggttgcgggccgggcctgggccgggtccgggccgggtattgcccgcctaatgagcgggcttttttt
tcttaccccttcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggc
cagcaaaaggccaggaaccgtaaaaaggccgcgttgctggcgtttttccataggctccgcccccctgacgagca
tcacaaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttcggga
agcgtggcgctttctcatagctcacgctgtaggtatctcagttcggtgtaggtcgttcgctccaagctgggctg
tgtgcacgaaccccccgttcagcccgaccgctgcgccttatccggtaactatgtcttgagtccaacccggtaag
acacgacttatcgccactggcagcagccactggtaacaggattagcagagcgaggtatgtaggcggtgctacag
agttcttgaagtggtggcctaactacggctacactagaagaacagtatttggtatctgcgctctgctgaagcca
gttaccttcggaaaaagagttggtagctcttgatccggcaaacaaaccaccgctggtagcggtggtttttttgt
ttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcac
ctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcct
gactcctgcaaaccacgttgtggtagaattggtaaagagagtcgtgtaaaatatcgagttcgcacatcttg
ttgtctgattattgatttttggcgaaaccatttgatcatatgacaagatgtgtatctaccttaacttaa
tgattttgataaaaatcattaggtaccccggcccgcactgacccctggtgttgctttttttttttaggccg
caagctgaagcgtgtccactagctgactagctaaccgcggaaacagTTACCAATGCTTAATCAGTGAGGCAC
CTATCTCAGCGATCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCATAATTCTC
TTACTGTCATGCCATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACCGCTGTTG
AGATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAATTTCCCCG
AAAtccctatcagtgatagagaaaagtgaaagtcgagtttaccactccctatcagtgatagagaaaagtgaaag
tcgagtttaccactccctatcagtgatagagaaaagtgaaagtcgagtttaccactccctatcagtgatagaga
aaagtgaaagtcgagtttaccactccctatcagtgatagagaaaagtgaaagtcgagtttaccactccctatca
gtgatagagaaaagtgaaagtcgagtttaccactccctatcagtgatagagaAGTGCCACCTGACGTCTAAGAA
ACCATTATTATCATGACATTAACCTATAAAAATAGGCGTATCACGAGGCCCTTTCGTC
SEQ ID NO: 7
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGG
GCTGGCTTAACTATGCGGCATCAGAGCAGATTGTACTGAGAGTGCACCATATGCGGTGTGAAATACCG
CACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAG
GCGATTAAGTTGGGTAACGCCAGGGTTTTCCCAGTCACGACGTTGTAAAACGACGGCCAGTGAATT
CGAGCTCGGTACCTCGCGAATGCATCTAGAgcggccgcataacttcgtatagcatacattatacgaacggta
ACCTGGGTACCCATAGAGCCCACCGCATCCCCAGCATGCCTGCTATTGTCTTCCCAATCCTCCCCCTTG
CTGTCCTGCCCCACCCCACCCCCCAGAATAGAATGACACCTACTCAGACAATGCGATGCAATTTCCTCA
TTTTATTAGGAAAGGACAGTGGGAGTGGCACCTTCCAGGGTCAAGGAAGGCACGGGGGAGGGGCAAA
CAACAGATGGCTGGCAACTAGAAGGCACAGCAGATCTGATCCTCATCAcaggtgcagctgcaggaaagcggccc
gggcctggtgaaaccgagcgaaaccctgagcgtgacctgcagcgtgagcggcgatagcatgaacaactattatt
ggacctggattcgccagagcccgggcaaaggcctggaatggattggctatattagcgatcgcgaaagcgcgacc
tataacccgagcctgaacagccgcgtggtgattagccgcgataccagcaaaaaccagctgagcctgaaactgaa
cagcgtgaccccggcggataccgcggtgtattattgcgcgaccgcgcgccgcggccagcgcatttatggcgtgg
tgagctttggcgaatttttttattattatagcatggatgtgtggggcaaaggcaccaccgtgaccgtgagcagc
gcgagcaccaaaggcccgagcgtgtttccgctggcgccgagcagcaaaagcaccagcggcggcaccgcggcgct
gggctgcctggtgaaagattattttccggaaccggtgaccgtgagctggaacagcggcgcgctgaccag
cggcgtgcatacctttccggcggtgctgcagagcagcggcctgtatagcctgagcagcgtggtgaccgtgc
cgagcagcagcctgggcacccagacctatatttgcaacgtgaaccataaaccgagcaacaccaaagtggataaa
cgcgtggaaccgaaaagctgcgataaaaccAGAATGTACACCGGTTGCAGTTGCTACTAGAAAAAGGATGATAC
ATGACCATCCCATGGTGGTCTAGtGCaGttaCGATATCTGATCACACGTGTCGAGGACaGTGACTGCAGAT
TGCTTTGAATTAGCGGTGGTTTTCACAACACCTAAAAAAGGGTTTAAAAGATACCTTTGAACCGCTAA
GAAGCCCGAGAATTAGCTCCGCTCAAAACTCAAGGGGACAAATTCCAAAAATGACTTCCAGCGCCAG
GCTGGCCTGACTAGTCTCCACCCACCAAATGTGAACAAACTCCAACGCCATTACATCCCCTCCCCCCGC
CGCGACTAGCCGTGCTCAAAAGCCCGAGGTGACTATTGCGGCCGATAGGACCACGGGGTCACAGGAA
GCAGCAGCCGGTGAGGGACCAGGCCCTCTTCCTTTGTGTGGGTGACTCACCCGCCCGCTCCACCGGGC
TGCCGCGTCCTCCATTTTGAGCTCCTTGCAACAGGGCCCGGGAGCGGCCATCTTTCCACGCACGCAACT
GGTGCCGGACGGGATGGCCTCACCCTAGTTAGGGAGGCAGGGCAACGCGGCGCCGCCAAGCCAGATC
GTGCCGGGTGCTGGGGCCACATGGCCTCGGCACGCTAACCCCAGCCTGGTTGCTTCGGGAAAAACCCC
AGGCCTCGCCCCATCCAGGTGGCGTCGGACATGTGCTCCGAAGGCGGGCGGGCCCCAGCCGCCACTCC
TGTCCCTCCATTCCTCCCCAACCATGACCTCTCCGGGCTCCGGGCGAGCAAGCCCCGCACCCTCCCTTT
GTTAGCCCCTATTGCTGAACGGCAATCGAAGGCAGCAGGGCAACAACAACAAAAAAAAAAAAGACCA
GAGTGCGGCCGGAGTAGCACGCGGCGGCGGCGCGGACACCACGCTAGGCCTCAAGCCGGACACGAGG
CGAGGCTACGGGGTTGCCGCTAGGCCTCGCACTCTGCCTCCCGCGCCGCCCGCAACTCGAAGCGGGAA
TGCTCGCAGCTAATCCCCGCCGACGACAGCGGGGCCCGGCCCGCTCGGAGCAGGACCTCCAGCTCGGC
GGCCCGCGGAAGCCACACCCGCCCCTCACCTGCGTTCTGACGGCAAACCCGTTGCGAAAAAGAACGTC
CAAGGCGACTGCCGCACTTATATACCGTTCTCCCCCACCCTCGGGAAGAGGGCGGAGCCAGCACACGA
CACCGCTTTCCCAGTTTGCCCCGCGCCAGCTCCCCGGTCCTCCCCCCCACGACCAGGGTCTATGAGCTT
GTGTGCTCTGCTCTCCCCGCGCTCAGCCCTGAGCGCATGCTCCTCGCGCTGTCGTGGGGCGGCTACAGG
GATTTGGGTCTGTGAGAATTTAGGGACGGTCCCTGGGCTCTCCCCTTGCGAAGGCGATCCCTCCTTTTG
TATGAATTACTCTCGGCTCCACTAGTGGGCCCTGCAaGgaattctGTTAGGGTGTGGAAAGTCCCCAGGCTC
CCCAGCAGGCAGAAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATAGTCCcctaggGAGA
CATGTTTACATAACTTACGGTAAATGGCCCGCCTGGCTGACCGCCCAACGACCCCCGCCCATTGACGTC
AATAATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGGCAGT
ACATCTACGTATTAGTCATCGCTATTACCATGAATTGGTTTGATCTGATTATAACTAGTGGAGAAGAGC
ATGCTTGAGGGCTGAGTGCCCCTCAGTGGGCAGAGAGCACATGGCCCACAGTCCCTGAGAAGTTGGGG
GGAGGGGTGGGCAATTGAACTGGTGCCTAGAGAAGGTGGGGCTTGGGTAAACTGGGAAAGTGATGTG
GTGTACTGGCTCCACCTTTTTCCCCAGGGTGGGGGAGAACCATATATAAGTGCAGTAGTCTCTGTGAAC
ATTCGCTAGCACACGTGTGATCAGATATCatGGCtGCTCTtAGACCACCATGGGATGGTCATGTATCATCC
TTTTTCTAGTAGCAACTGCAACCGGTGTACATTCAagctatgtgcgcccgctgagcgtggcgctgggcgaaacc
gcgcgcattagctgcggccgccaggcgctgggcagccgcgcggtgcagtggtatcagcatcgcccgggccaggc
gccgattctgctgatttataacaaccaggatcgcccgagcggcattccggaacgctttagcggcaccccggata
ttaactttggcacccgcgcgaccctgaccattagcggcgtggaagcgggcgatgaagcggattattattgccat
atgtgggatagccgcagcggctttagctggagctttggcggcgcgacccgcctgaccgtgctgggccagccgaa
agcggcgccgagcgtgaccctgtttccgccgagcagcgaagaactgcaggcgaacaaagcgaccctggtgtgcc
tgattagcgatttttatccgggcgcggtgaccgtggcgtggaaagcggatagcagcccggtgaaagcg
ggcgtggaaaccaccaccccgagcaaacagagcaacaacaaatatgcggcgagcagctatctgagc
ctgaccccggaacagtggaaaagccatcgcagctatagctgccaggtgacccatgaaggcagcaccg
tggaaaaaaccgtggcgccgaccgaatgcagcTGATAGGATaagctttGATCTAAATAAACTTATAAAT
TGTGAGAGAAATTAATGAATGTCTAAGTTAATGCAGAAACGGAGAGACATACTATATTCATGAACTAA
AAGACTTAATATTGTGAAGGTATACTTTCTTTCCACATAAATTTGTAGTCAATATGTTCACCCCAAAAA
AGCTGTTTGTTAACTTGCCAACCTCATTCTAAAATGTATATAGAAGCCCAAAAGACAATAACAAAAAT
ATTCTTGTAGAACAAAATGGGAAAGAATGTTCCACTAAATATCAAGATTTAGAGCAAAGCATGAGATG
TGTGGGGATAGACAGTGAGGCTGATAAAATAGAGTAGAGCTCAGAAACAGACCCATTGATATATGTA
AGTGACCTATGAAAAAAATATGGCATTTTACAATGGGAAAATGATGATCTTTTTCTTTTTTAGAAAAAC
AGGGAAATATATTTATATGTAAAAAATAAAAGGGAACCCATATGTCATACCATACACACAAAAAAATT
CCAGTGAATTATAAGTCTAAATGGAGAAGGCAAAACTTTAAATCTTTTAGAAAATAATATAGAAGCAT
GCCATCATGACTTCAGTGTAGAGAAAAATTTCTTATGACTCAAAGTCCTAACCACAAAGAAAAGATTG
TTAATTAGATTGCATGAATATTAAGACTTATTTTTAAAATTAAAAAACCATTAAGAAAAGTCAGGCCAT
AGAATGACAGAAAATATTTGCAACACCCCAGTAAAGAGAATTGTAATATGCAGATTATAAAAAGAAG
TCTTACAAATCAGTAAAAAATAAAACTAGACAAAAATTTGAACAGATGAAAGAGAAACTCTAAATAA
TCATTACACATGAGAAACTCAATCTCAGAAATCAGAGAACTATCATTGCATATACACTAAATTAGAGA
AATATTAAAAGGCTAAGTAACATCTGTGGCAATATTGATGGTATATAACCTTGATATGATGTGATGAG
AACAGTACTTTACCCCATGGGCTTCCTCCCCAAACCCTTACCCCAGTATAAATCATGACAAATATACTT
TAAAAACCATTACCCTATATCTAACCAGTACTCCTCAAAACTGTCAAGGTCATCAAAAATAAGAAAAG
TCTGAGGAACTGTCAAAACTAAGAGGAACCCAAGGAGACATGAGAATTATATGTAATGTGGCATTCTG
AATGAGATCCCAGAACAGAAAAAGAACAGTAGCTAAAAAACTAATGAAATATAAATAAAGTTTGAAC
TTTAGTTTTTTTTAAAAAAGAGTAGCATTAACACGGCAAAGCCATTTTCATATTTTTCTTGAACATTAA
GTACAAGTCTATAATTAAAAATTTTTTAAATGTAGTCTGGAACATTGCCAGAAACAGAAGTACAGCAG
CTATCTGTGCTGTCGCCTAACTATCCATAGCTGATTGGTCTAAAATGAGATACATCAACGCTCCTCCAT
GTTTTTTGTTTTCTTTTTAAATGAAAAACTTTATTTTTTAAGAGGAGTTTCAGGTTCATAGCAAAATTGA
GAGGAAGGTACATTCAAGCTGAGGAAGTTTTCCTCTATTCCTAGTTTACTGAGAGATTGCATCATGAAT
GGGTGTTAAATTTTGTCAAATGCTTTTTCTGTGTCTATCAATATGACCATGTGATTTTCTTCTTTAACCT
GTTGATGGGACAAATTACGTTAATTGATTTTCAAACGTTGAACCACCCTTACATATCTGGAATAAATTC
TACTTGGTTGTGGTGTATATTTTTTGATACATTCTTGGATTCTTTTTGCTAATATTTTGTTGAAAATGTTT
GTATCTTTGTTCATGAGAGATATTGGTCTGTTGTTTTCTTTTCTTGTAATGTCATTTTCTAGTTCCGGTAT
TAAGGTAATGCTGGCCTAGTTGAATGATTTAGGAAGTATTCCCTCTGCTTCTGTCTTCTGAAAGAGATT
GTAGAAAGTTGATACAATTTTTTTTTCTTTAAATATCTTGATAGAATTggatcctatcaatcGTCGACtGTACC
ttGGGCCtGGGACCGGAACTGAAACATAAAATGAATGCAAcTGTTGTTGTTAACTTGTTTATTGCAGCTTAT
AATGGTTACAAATAAAGCAATAGCATCACAAATTTCACAAATAAAGCATTTTTTTCACTGCATTCTAGT
TGTGGTTTGTCCAAACTCATCAATGTATCTTATCATGTCTGGATCACGCGTAGtctagataccgttcgtatagc
atacattatacgaagttatgtttaaacagCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTG
CGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTCTAAGAAA
CCATTATTATCATGACATTAACCTATAAAAATAGGCGTATCACGAGGCCCTTTCGTC

### Exemplary Amino Acid Sequences of Antibodies Disclosed Herein

| Chain H, 10E8v4-5R + 100cF Fab heavy chain | |
|---|---|
| 1 | evrlresggg lvkpggslrl scsasgfdfd nawmtwvrqp pgkglewvgr itgpgegwsv |
| 61 | dyaesvkgrf tisrdntknt lylemnnvrt edtgyyfcar tgkyydfwfg yppgeeyfqd |
| 121 | wgqgtlvivs sastkgpsvf plapssksts ggtaalgclv kdyfpepvtv swnsgalts |
| 181 | vhtfpavlqs sglyslssvv tvpssslgtq tyicnvnhkp sntkvdkkve pk |
| Chain L, FA10E8v4-5R + 100cF FAB light chain | |
| 1 | seltqdpavs valkqtvtit crgdslrshy aswyqkkpgq apvllfygkn nrpsgipdrf |
| 61 | sgsasgnras ltitgaqaed eadyycssrd ksgsrlsvfg ggtkltvlsq pkaapsvtlf |
| 121 | ppsseelqan katlvclisd fypgavtvaw kadsspvkag vetttpskqs nnkyaassyl |
| 181 | sltpeqwksh rsyscqvthe gstvektvap tecs |
| Chain A, 10E8v4-5R + 100cF Fab heavy chain | |
| 1 | evrlresggg lvkpggslrl scsasgfdfd nawmtwvrqp pgkglewvgr itgpgegwsv |
| 61 | dyaesvkgrf tisrdntknt lylemnnvrt edtgyyfcar tgkyydfwfg yppgeeyfqd |
| 121 | wgqgtlvivs sastkgpsvf plapssksts ggtaalgclv kdyfpepvtv swnsgaltsg |
| 181 | vhtfpavlqs sglyslssvv tvpssslgtq tyicnvnhkp sntkvdkkve pk |
| Chain B, FA10E8v4-5R + 100cF FAB light chain | |
| 1 | seltqdpavs valkqtvtit crgdslrshy aswyqkkpgq apvllfygkn nrpsgipdrf |
| 61 | sgsasgnras ltitgaqaed eadyycssrd ksgsrlsvfg ggtkltvlsq pkaapsvtlf |
| 121 | ppsseelqan katlvclisd fypgavtvaw kadsspvkag vetttpskqs nnkyaassyl |
| 181 | sltpeqwksh rsyscqvthe gstvektvap tecs |
| Chain D, Human Anti-hiv-1 Antibody Pgdm1400 Heavy Chain | |
| 1 | qaqlvqsgpe vrkpgtsvkv sckapgntlk tydlhwvrsv pgqglqwmgw ishegdkkvi |
| 61 | verfkakvti dwdrstntay lqlsgltsgd tavyycakgs khrlrdyalx dddgalnwav |
| 121 | dvdylsnlef wgqgtavtvs sastkgpsvf plapssksts ggtaalgclv kdyfpepvtv |
| 181 | swnsgaltsg vhtfpavlqs sglyslssvv tvpssslgtq tyicnvnhkp sntkvdkkve |
| 241 | pkscd |
| Chain C, Human Anti-hiv-1 Antibody Pgdm1400 Light Chain | |
| 1 | dfvltqsphs lsvtpgesas iscksshsli hgdrnnylaw yvqkpgrspq lliylassra |
| 61 | sgvpdrfsgs gsdkdftlki srvetedvgt yycmqgresp wtfgqgtkvd ikrtvaapsv |
| 121 | fifppsdeql ksgtasvvcl lnnfypreak vqwkvdnalq sgnsqesvte qdskdstysl |
| 181 | sstltlskad yekhkvyace vthqglsspv tksfnrgec |
| Chain B, Human Anti-hiv-1 Antibody Pgdm1400 Heavy Chain | |
| 1 | qaqlvqsgpe vrkpgtsvkv sckapgntlk tydlhwvrsv pgqglqwmgw ishegdkkvi |
| 61 | verfkakvti dwdrstntay lqlsgltsgd tavyycakgs khrlrdyalx dddgalnwav |
| 121 | dvdylsnlef wgqgtavtvs sastkgpsvf plapssksts ggtaalgclv kdyfpepvtv |
| 181 | swnsgaltsg vhtfpavlqs sglyslssvv tvpssslgtq tyicnvnhkp sntkvdkkve |
| 241 | pkscd |
| Chain A, Human Anti-hiv-1 Antibody Pgdm1400 Light Chain | |
| 1 | dfvltqsphs lsvtpgesas iscksshsli hgdrnnylaw yvqkpgrspq lliylassra |
| 61 | sgvpdrfsgs gsdkdftlki srvetedvgt yycmqgresp wtfgqgtkvd ikrtvaapsv |
| 121 | fifppsdeql ksgtasvvcl lnnfypreak vqwkvdnalq sgnsqesvte qdskdstysl |
| 181 | sstltlskad yekhkvyace vthqglsspv tksfnrgec |
| Chain H, Human Anti-hiv-1 Antibody Pgdm1400 Heavy Chain | |
| 1 | qaqlvqsgpe vrkpgtsvkv sckapgntlk tydlhwvrsv pgqglqwmgw ishegdkkvi |
| 61 | verfkakvti dwdrstntay lqlsgltsgd tavyycakgs khrlrdyalx dddgalnwav |
| 121 | dvdylsnlef wgqgtavtvs sastkgpsvf plapssksts ggtaalgclv kdyfpepvtv |
| 181 | swnsgaltsg vhtfpavlqs sglyslssvv tvpssslgtq tyicnvnhkp sntkvdkkve |
| 241 | pkscd |
| Chain L, Human Anti-hiv-1 Antibody Pgdm1400 Light Chain | |
| 1 | dfvltqsphs lsvtpgesas iscksshsli hgdrnnylaw yvqkpgrspq lliylassra |
| 61 | sgvpdrfsgs gsdkdftlki srvetedvgt yycmqgresp wtfgqgtkvd ikrtvaapsv |
| 121 | fifppsdeql ksgtasvvcl lnnfypreak vqwkvdnalq sgnsqesvte qdskdstysl |
| 181 | sstltlskad yekhkvyace vthqglsspv tksfnrgec |
| Chain L, Light Chain Of Antibody N6 | |
| 1 | yihvtqspss lsvsigdrvt incqtsqgvg sdlhwyqhkp grapkllihh tssvedgvps |
| 61 | rfsgsgfhts fnltisdlqa ddiatyycqv lqffgrgsrl hikrtvaaps vfifppsdeq |
| 121 | lksgtasvvc llnnfyprea kvqwkvdnal qsgnsqesvt eqdskdstys lsstltlska |
| 181 | dyekhkvyac evthqglssp vtksfnrgec |
| Chain H, Heavy Chain Of Antibody N6 | |
| 1 | rahlvqsgta mkkpgasvrv scqtsgytft ahilfwfrqa pgrglewvgw ikpqygavnf |
| 61 | gggfrdrvtl trdvyreiay mdirglkpdd tavyycardr sygdsswald awgqgttvvv |
| 121 | saastkgpsv fplapsskst sggtaalgcl vkdyfpepvt vswnsgalts gvhtfpavlq |
| 181 | ssglyslssv vtvpssslgt qtyicnvnhk psntkvdkkv epksc |

1.-64. (canceled)

65. A nucleic acid encoding an HIV specific antibody, wherein the nucleic acid is a minicircle plasmid or a mini-intronic plasmid.

66. The nucleic acid of claim 65, wherein the HIV specific antibody is a Fab, F(ab)₂, a single-domain antibody, or a single chain variable fragment (scFv).

67. The nucleic acid of claim 65, wherein the HIV specific antibody is an IgG antibody.

68. The nucleic acid of claim 65, wherein the HIV specific antibody is a broadly neutralizing antibody.

69. The nucleic acid of claim 65, wherein the HIV specific antibody binds to HIV gp120 and/or gp41.

70. The nucleic acid of claim 68, wherein the broadly neutralizing antibody comprises 10E8.4, N6-LS, or PGDM1400.

71. The nucleic acid of claim 68, wherein the broadly neutralizing antibody comprises 2F5, 4E10, 2G12, or B12.

72. The nucleic acid of claim 68, wherein the broadly neutralizing antibody comprises N6-LS.

73. The nucleic acid of claim 68, wherein the broadly neutralizing antibody comprises VRC07-523, CAP256-VRC26.25, 10-1074V, or 10E.

74. The nucleic acid of claim 68, wherein the broadly neutralizing antibody comprises VRC07, VRC01, VRC13, CAP256-VRC26.08, PGT145, PG9, PGT121, or PGT128.

75. The nucleic acid of claim 65, wherein the minicircle plasmid or the mini-intronic plasmid comprises a S/MAR region.

76. A complex comprising the nucleic acid of claim 65 complexed to a cationic lipid, a cationic polymer, or a nanodiamond.

77. The complex of claim 76, wherein the nucleic acid of claim 65 is complexed to a nanodiamond.

78. The complex of claim 76, wherein the cationic lipid or cationic polymer comprises polyethylenimine, polylysine (PLL), PEG-grafted-polylysine (PEG-g-PLL), DOTMA, DOGS, DC-Chol, DEAE-dextran, DOSPA, DOPE, DOTAP, or combinations thereof.

79. The complex of claim 78, wherein the cationic lipid or cationic polymer comprises polyethylenimine, polylysine (PLL), or PEG-grafted-polylysine (PEG-g-PLL).

80. A composition comprising the nucleic acid of claim 65 and a pharmaceutically acceptable carrier, diluent, or excipient.

81. A method of treating HIV infection in an individual comprising administering the nucleic acid of claim 65 to the individual.

82. A composition comprising a first nucleic acid and a second nucleic acid, wherein the first nucleic acid encodes a first HIV specific antibody that binds an HIV polypeptide, wherein the second nucleic acid encodes a second HIV specific antibody that binds an HIV polypeptide, wherein the amino acid sequence of the first HIV antibody is different from the amino acid sequence of the second HIV antibody.

83. The composition according to claim 82, wherein the composition comprises a third nucleic acid that encodes a third HIV specific antibody that specifically binds an HIV polypeptide, wherein the amino acid sequence of the third HIV antibody is different from the amino acid sequence of the first and second HIV antibody.

84. The composition of claim 83, wherein any one or more of the first HIV specific antibody, second HIV specific antibody, or the third HIV specific antibody is a Fab, F(ab)₂, a single-domain antibody, or a single chain variable fragment (scFv).


SEQUENCE LISTING (14 sequences)

SEQ ID NO: 1 - DNA, 9085 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcatttacctggagacagggagaggctcttctgcgtgtagtggttgtgcagagcctcatgcatcacggagcatgagaagacgttcccctgctgccacctgctcttgtccacggtgagcttgctgtagaggaagaaggagccatcagagtccagcacgggaggcgtggtcttgtagttgttctccggctgcccattgctctcccactccacagcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttgctgaccttgcacttgtactccttgccattcagccagtcctggtgcaggacggtgaggacactgaccacacggtacgtgctgttgtactgctcctccctcggctttgtcttggcattatgcacctccactccatccacgtaccagttgaacttgacctcagggtcttcatggctcacatccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggctcctgagttccacgacacagtcaccggttcggggaagtagtccttgaccaggcagcccagggccgctgtgcccccagaggtgctcttggaggagggtgccagggggaagacacttgggcccttggtactcgccgcagagacgacgacagtcgttccctgtccccaggcatctaaggcccaagaggagtctccataggacctatctctcgcacagtaatagacagccgtgtcatcaggtttaaggcctctgatgtccatgtacgcaatctctctatatacatctcgtgtcaatgtgaccctgtcccgaaaaccaccaccaaaattcacagccccatattgtggcttgatccaccccacccactcaagtcctcgcccaggggcctgtcggaaccaaaataatatgtgggcggtaaaggtgtatccagaggtctggcaggagactcttactgaggctcccggtttcttcatcgcagtccctgattgtaccaggtgcgctcgagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcatacatccacgtgacccagtctccgtcctccctgtctgtgtctattggagacagagtcaccatcaactgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaattctttggccgagggagtagactccatattaaacgtacggtggctgcaccatctgtcttcatcttcccaccatctgatgagcagttgaaatctggaactgcctctgttgtgtgcctgctgaataacttctaccccagagaagccaaagtgcagtggaaggtggacaatgccctccagagcggaaacagccaggaatctgtgacagagcaggattccaaggattccacatacagcctgagcagcacactgacactgtccaaggctgactacgagaagcacaaggtgtacgcctgcgaagtgacacaccagggactgtcctcccctgtgacaaagagcttcaacagaggagaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatgtttaaacagctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 9711 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcatttacctggagacagggagaggctcttctgcgtgtagtggttgtgcagagcctcatgcatcacggagcatgagaagacgttcccctgctgccacctgctcttgtccacggtgagcttgctgtagaggaagaaggagccatcagagtccagcacgggaggcgtggtcttgtagttgttctccggctgcccattgctctcccactccacagcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttgctgaccttgcacttgtactccttgccattcagccagtcctggtgcaggacggtgaggacactgaccacacggtacgtgctgttgtactgctcctccctcggctttgtcttggcattatgcacctccactccatccacgtaccagttgaacttgacctcagggtcttcatggctcacatccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggctcctgagttccacgacacagtcaccggttcggggaagtagtccttgaccaggcagcccagggccgctgtgcccccagaggtgctcttggaggagggtgccagggggaagacacttgggcccttggtactcgccgcagagacgacgacagtcgttccctgtccccaggcatctaaggcccaagaggagtctccataggacctatctctcgcacagtaatagacagccgtgtcatcaggtttaaggcctctgatgtccatgtacgcaatctctctatatacatctcgtgtcaatgtgaccctgtcccgaaaaccaccaccaaaattcacagccccatattgtggcttgatccaccccacccactcaagtcctcgcccaggggcctgtcggaaccaaaataatatgtgggcggtaaaggtgtatccagaggtctggcaggagactcttactgaggctcccggtttcttcatcgcagtccctgattgtaccaggtgcgctcgagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcatacatccacgtgacccagtctccgtcctccctgtctgtgtctattggagacagagtcaccatcaactgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaattctttggccgagggagtagactccatattaaacgtacggtggctgcaccatctgtcttcatcttcccaccatctgatgagcagttgaaatctggaactgcctctgttgtgtgcctgctgaataacttctaccccagagaagccaaagtgcagtggaaggtggacaatgccctccagagcggaaacagccaggaatctgtgacagagcaggattccaaggattccacatacagcctgagcagcacactgacactgtccaaggctgactacgagaagcacaaggtgtacgcctgcgaagtgacacaccagggactgtcctcccctgtgacaaagagcttcaacagaggagaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatgtttcggccggtggatccaccggtattgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtccactagctgactagctaaccgcggaaacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaatttccccgaaaagtgccacctgacgtctaagaaaccattattatcatgacattaacctataaaaataggcgtatcacgaggccctttcgtc

SEQ ID NO: 3 - DNA, 9982 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcatttacctggagacagggagaggctcttctgcgtgtagtggttgtgcagagcctcatgcatcacggagcatgagaagacgttcccctgctgccacctgctcttgtccacggtgagcttgctgtagaggaagaaggagccatcagagtccagcacgggaggcgtggtcttgtagttgttctccggctgcccattgctctcccactccacagcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttgctgaccttgcacttgtactccttgccattcagccagtcctggtgcaggacggtgaggacactgaccacacggtacgtgctgttgtactgctcctccctcggctttgtcttggcattatgcacctccactccatccacgtaccagttgaacttgacctcagggtcttcatggctcacatccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggctcctgagttccacgacacagtcaccggttcggggaagtagtccttgaccaggcagcccagggccgctgtgcccccagaggtgctcttggaggagggtgccagggggaagacacttgggcccttggtactcgccgcagagacgacgacagtcgttccctgtccccaggcatctaaggcccaagaggagtctccataggacctatctctcgcacagtaatagacagccgtgtcatcaggtttaaggcctctgatgtccatgtacgcaatctctctatatacatctcgtgtcaatgtgaccctgtcccgaaaaccaccaccaaaattcacagccccatattgtggcttgatccaccccacccactcaagtcctcgcccaggggcctgtcggaaccaaaataatatgtgggcggtaaaggtgtatccagaggtctggcaggagactcttactgaggctcccggtttcttcatcgcagtccctgattgtaccaggtgcgctcgagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcatacatccacgtgacccagtctccgtcctccctgtctgtgtctattggagacagagtcaccatcaactgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaattctttggccgagggagtagactccatattaaacgtacggtggctgcaccatctgtcttcatcttcccaccatctgatgagcagttgaaatctggaactgcctctgttgtgtgcctgctgaataacttctaccccagagaagccaaagtgcagtggaaggtggacaatgccctccagagcggaaacagccaggaatctgtgacagagcaggattccaaggattccacatacagcctgagcagcacactgacactgtccaaggctgactacgagaagcacaaggtgtacgcctgcgaagtgacacaccagggactgtcctcccctgtgacaaagagcttcaacagaggagaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatgtttcggccggtggatccaccggtattgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtccactagctgactagctaaccgcggaaacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaatttccccgaaatccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaagtgccacctgacgtctaagaaaccattattatcatgacattaacctataaaaataggcgtatcacgaggccctttcgtc

SEQ ID NO: 4 - DNA, 8491 bp, Artificial Sequence
  Description of Artificial Sequence Synthetic polynucleotide
  modified_base (1053)..(1055): a, c, t, g, unknown or other
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcacaggcgcagctggtgcagagcggcccggaagtgcgcaaaccgggcaccagcgtgaaagtgagctgcaaagcgccgggcaacaccctgaaaacctatgatctgcattgggtgcgcagcgtgccgggccagggcctgcagtggatgggctggattagccatgaaggcgataaaaaagtgattgtggaacgctttaaagcgaaagtgaccattgattgggatcgcagcaccaacaccgcgtatctgcagctgagcggcctgaccagcggcgataccgcggtgtattattgcgcgaaaggcagcaaacatcgcctgcgcgattatgcgctgnnngatgatgatggcgcgctgaactgggcggtggatgtggattatctgagcaacctggaattttggggccagggcaccgcggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgatagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcagattttgtgctgacccagagcccgcatagcctgagcgtgaccccgggcgaaagcgcgagcattagctgcaaaagcagccatagcctgattcatggcgatcgcaacaactatctggcgtggtatgtgcagaaaccgggccgcagcccgcagctgctgatttatctggcgagcagccgcgcgagcggcgtgccggatcgctttagcggcagcggcagcgataaagattttaccctgaaaattagccgcgtggaaaccgaagatgtgggcacctattattgcatgcagggccgcgaaagcccgtggacctttggccagggcaccaaagtggatattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatgtttaaacagctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 9131 bp, Artificial Sequence
  Description of Artificial Sequence Synthetic polynucleotide
  modified_base (1053)..(1055): a, c, t, g, unknown or other
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcacaggcgcagctggtgcagagcggcccggaagtgcgcaaaccgggcaccagcgtgaaagtgagctgcaaagcgccgggcaacaccctgaaaacctatgatctgcattgggtgcgcagcgtgccgggccagggcctgcagtggatgggctggattagccatgaaggcgataaaaaagtgattgtggaacgctttaaagcgaaagtgaccattgattgggatcgcagcaccaacaccgcgtatctgcagctgagcggcctgaccagcggcgataccgcggtgtattattgcgcgaaaggcagcaaacatcgcctgcgcgattatgcgctgnnngatgatgatggcgcgctgaactgggcggtggatgtggattatctgagcaacctggaattttggggccagggcaccgcggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgatagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcagattttgtgctgacccagagcccgcatagcctgagcgtgaccccgggcgaaagcgcgagcattagctgcaaaagcagccatagcctgattcatggcgatcgcaacaactatctggcgtggtatgtgcagaaaccgggccgcagcccgcagctgctgatttatctggcgagcagccgcgcgagcggcgtgccggatcgctttagcggcagcggcagcgataaagattttaccctgaaaattagccgcgtggaaaccgaagatgtgggcacctattattgcatgcagggccgcgaaagcccgtggacctttggccagggcaccaaagtggatattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatacgcgtagtctagagtttcggccggtggatccaccggtattgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtccactagctgactagctaaccgcggaaacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaatttccccgaaaagtgccacctgacgtctaagaaaccattattatcatgacattaacctataaaaataggcgtatcacgaggccctttcgtc

SEQ ID NO: 6 - DNA, 9402 bp, Artificial Sequence
  Description of Artificial Sequence Synthetic polynucleotide
  modified_base (1053)..(1055): a, c, t, g, unknown or other
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcacaggcgcagctggtgcagagcggcccggaagtgcgcaaaccgggcaccagcgtgaaagtgagctgcaaagcgccgggcaacaccctgaaaacctatgatctgcattgggtgcgcagcgtgccgggccagggcctgcagtggatgggctggattagccatgaaggcgataaaaaagtgattgtggaacgctttaaagcgaaagtgaccattgattgggatcgcagcaccaacaccgcgtatctgcagctgagcggcctgaccagcggcgataccgcggtgtattattgcgcgaaaggcagcaaacatcgcctgcgcgattatgcgctgnnngatgatgatggcgcgctgaactgggcggtggatgtggattatctgagcaacctggaattttggggccagggcaccgcggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgatagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcagattttgtgctgacccagagcccgcatagcctgagcgtgaccccgggcgaaagcgcgagcattagctgcaaaagcagccatagcctgattcatggcgatcgcaacaactatctggcgtggtatgtgcagaaaccgggccgcagcccgcagctgctgatttatctggcgagcagccgcgcgagcggcgtgccggatcgctttagcggcagcggcagcgataaagattttaccctgaaaattagccgcgtggaaaccgaagatgtgggcacctattattgcatgcagggccgcgaaagcccgtggacctttggccagggcaccaaagtggatattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatacgcgtagtctagagtttcggccggtggatccaccggtattgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtccactagctgactagctaaccgcggaaacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaatttccccgaaatccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaagtgccacctgacgtctaagaaaccattattatcatgacattaacctataaaaataggcgtatcacgaggccctttcgtc

SEQ ID NO: 7 - DNA, 8455 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcggccgcataacttcgtatagcatacattatacgaacggtaacctgggtacccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcagatctgatcctcatcacaggtgcagctgcaggaaagcggcccgggcctggtgaaaccgagcgaaaccctgagcgtgacctgcagcgtgagcggcgatagcatgaacaactattattggacctggattcgccagagcccgggcaaaggcctggaatggattggctatattagcgatcgcgaaagcgcgacctataacccgagcctgaacagccgcgtggtgattagccgcgataccagcaaaaaccagctgagcctgaaactgaacagcgtgaccccggcggataccgcggtgtattattgcgcgaccgcgcgccgcggccagcgcatttatggcgtggtgagctttggcgaatttttttattattatagcatggatgtgtggggcaaaggcaccaccgtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaacgcgtggaaccgaaaagctgcgataaaaccagaatgtacaccggttgcagttgctactagaaaaaggatgatacatgaccatcccatggtggtctagtgcagttacgatatctgatcacacgtgtcgaggacagtgactgcagattgctttgaattagcggtggttttcacaacacctaaaaaagggtttaaaagatacctttgaaccgctaagaagcccgagaattagctccgctcaaaactcaaggggacaaattccaaaaatgacttccagcgccaggctggcctgactagtctccacccaccaaatgtgaacaaactccaacgccattacatcccctccccccgccgcgactagccgtgctcaaaagcccgaggtgactattgcggccgataggaccacggggtcacaggaagcagcagccggtgagggaccaggccctcttcctttgtgtgggtgactcacccgcccgctccaccgggctgccgcgtcctccattttgagctccttgcaacagggcccgggagcggccatctttccacgcacgcaactggtgccggacgggatggcctcaccctagttagggaggcagggcaacgcggcgccgccaagccagatcgtgccgggtgctggggccacatggcctcggcacgctaaccccagcctggttgcttcgggaaaaaccccaggcctcgccccatccaggtggcgtcggacatgtgctccgaaggcgggcgggccccagccgccactcctgtccctccattcctccccaaccatgacctctccgggctccgggcgagcaagccccgcaccctccctttgttagcccctattgctgaacggcaatcgaaggcagcagggcaacaacaacaaaaaaaaaaaagaccagagtgcggccggagtagcacgcggcggcggcgcggacaccacgctaggcctcaagccggacacgaggcgaggctacggggttgccgctaggcctcgcactctgcctcccgcgccgcccgcaactcgaagcgggaatgctcgcagctaatccccgccgacgacagcggggcccggcccgctcggagcaggacctccagctcggcggcccgcggaagccacacccgcccctcacctgcgttctgacggcaaacccgttgcgaaaaagaacgtccaaggcgactgccgcacttatataccgttctcccccaccctcgggaagagggcggagccagcacacgacaccgctttcccagtttgccccgcgccagctccccggtcctcccccccacgaccagggtctatgagcttgtgtgctctgctctccccgcgctcagccctgagcgcatgctcctcgcgctgtcgtggggcggctacagggatttgggtctgtgagaatttagggacggtccctgggctctccccttgcgaaggcgatccctccttttgtatgaattactctcggctccactagtgggccctgcaaggaattctgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccctagggagacatgtttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcgctagcacacgtgtgatcagatatcatggctgctcttagaccaccatgggatggtcatgtatcatcctttttctagtagcaactgcaaccggtgtacattcaagctatgtgcgcccgctgagcgtggcgctgggcgaaaccgcgcgcattagctgcggccgccaggcgctgggcagccgcgcggtgcagtggtatcagcatcgcccgggccaggcgccgattctgctgatttataacaaccaggatcgcccgagcggcattccggaacgctttagcggcaccccggatattaactttggcacccgcgcgaccctgaccattagcggcgtggaagcgggcgatgaagcggattattattgccatatgtgggatagccgcagcggctttagctggagctttggcggcgcgacccgcctgaccgtgctgggccagccgaaagcggcgccgagcgtgaccctgtttccgccgagcagcgaagaactgcaggcgaacaaagcgaccctggtgtgcctgattagcgatttttatccgggcgcggtgaccgtggcgtggaaagcggatagcagcccggtgaaagcgggcgtggaaaccaccaccccgagcaaacagagcaacaacaaatatgcggcgagcagctatctgagcctgaccccggaacagtggaaaagccatcgcagctatagctgccaggtgacccatgaaggcagcaccgtggaaaaaaccgtggcgccgaccgaatgcagctgataggataagctttgatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattggatcctatcaatcgtcgactgtaccttgggcctgggaccggaactgaaacataaaatgaatgcaactgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcacgcgtagtctagataccgttcgtatagcatacattatacgaagttatgtttaaacagctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - protein, 231 aa, Artificial Sequence
  Description of Artificial Sequence Synthetic polypeptide
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSASTKGPSVFPLAPSSKSTSGGTAALGCLVKDYFPEPVTVSWNSGALTSVHTFPAVLQSSGLYSLSSVVTVPSSSLGTQTYICNVNHKPSNTKVDKKVEPK

SEQ ID NO: 9 - protein, 214 aa, Artificial Sequence
  Description of Artificial Sequence Synthetic polypeptide
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 232 aa, Artificial Sequence
  Description of Artificial Sequence Synthetic polypeptide
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 11 - protein, 245 aa, Artificial Sequence
  Description of Artificial Sequence Synthetic polypeptide
  MOD_RES (110)..(110): Any amino acid
QAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALXDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 12 - protein, 219 aa, Artificial Sequence
  Description of Artificial Sequence Synthetic polypeptide
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 210 aa, Artificial Sequence
  Description of Artificial Sequence Synthetic polypeptide
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 225 aa, Artificial Sequence
  Description of Artificial Sequence Synthetic polypeptide
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC